SEQUENCE LISTING (121 sequences)

SEQ ID NO: 1 - DNA, 126 bp, Artificial Sequence
  chemically treated genomic DNA (Homo sapiens)
gggattatttttataaggttcggaggtcgcgaggttttcgttggagtttcgtcgtcgtagttttcgttattagtgagtacgcgcggttcgcgttttcggggatggggtttagagtttttagtatgg

SEQ ID NO: 2 - DNA, 20 bp, Artificial Sequence
  chemically treated genomic DNA (Homo sapiens)
gggattatttttataaggtt

SEQ ID NO: 3 - DNA, 22 bp, Artificial Sequence
  chemically treated genomic DNA (Homo sapiens)
ccatactaaaaactctaaaccc

SEQ ID NO: 4 - DNA, 25 bp, Artificial Sequence
  chemically treated genomic DNA (Homo sapiens)
cccatccccaaaaacacaaaccaca

SEQ ID NO: 5 - DNA, 21 bp, Artificial Sequence
  chemically treated genomic DNA (Homo sapiens)
ttcgtcgtcgtagttttcgtt

SEQ ID NO: 6 - DNA, 18 bp, Artificial Sequence
  chemically treated genomic DNA (Homo sapiens)
tagtgagtacgcgcggtt

SEQ ID NO: 7 - DNA, 130 bp, Artificial Sequence
  chemically treated genomic DNA (Homo sapiens)
ggagtggaggaaattgagatttattgaggttacgtagtttgtttaaggttaagtttgggtgtttgtaatttttgttttgtgttaggttgttttttaggtgttaggtgagttttgagtatttgttgtgtgg

SEQ ID NO: 8 - DNA, 20 bp, Artificial Sequence
  chemically treated genomic DNA (Homo sapiens)
ggagtggaggaaattgagat

SEQ ID NO: 9 - DNA, 22 bp, Artificial Sequence
  chemically treated genomic DNA (Homo sapiens)
ccacacaacaaatactcaaaac

SEQ ID NO: 10 - DNA, 27 bp, Artificial Sequence
  chemically treated genomic DNA (Homo sapiens)
gtttaaggttaagtttgggtgtttgta

SEQ ID NO: 11 - DNA, 29 bp, Artificial Sequence
  chemically treated genomic DNA (Homo sapiens)
ttttgttttgtgttaggttgttttttagg

SEQ ID NO: 12 - DNA, 123 bp, Artificial Sequence
  chemically treated genomic DNA (Homo sapiens)
gggattatttttataaggttcggaggtcgcgaggttttcgttggagtttcgtcgtcgtagttttcgttattagtgagtacgcgcggttcgcgttttcggggatggggtttagagtttttagta

SEQ ID NO: 13 - DNA, 23 bp, Artificial Sequence
  chemically treated genomic DNA (Homo sapiens)
tactaaaaactctaaaccccatc

SEQ ID NO: 14 - DNA, 26 bp, Artificial Sequence
  chemically treated genomic DNA (Homo sapiens)
catccccaaaaacacaaaccacacat

SEQ ID NO: 15 - DNA, 25 bp, Artificial Sequence
  chemically treated genomic DNA (Homo sapiens)
ccatccccaaaaacacaaaccacac

SEQ ID NO: 16 - DNA, 25 bp, Artificial Sequence
  chemically treated genomic DNA (Homo sapiens)
cccatccccaaaaacacaaaccaca

SEQ ID NO: 17 - DNA, 25 bp, Artificial Sequence
  chemically treated genomic DNA (Homo sapiens)
tccccaaaaacacaaaccacacata

SEQ ID NO: 18 - DNA, 24 bp, Artificial Sequence
  chemically treated genomic DNA (Homo sapiens)
cccatccccaaaaacacaaaccac

SEQ ID NO: 19 - DNA, 28 bp, Artificial Sequence
  chemically treated genomic DNA (Homo sapiens)
catccccaaaaacacaaaccacacatac

SEQ ID NO: 20 - DNA, 27 bp, Artificial Sequence
  chemically treated genomic DNA (Homo sapiens)
atccccaaaaacacaaaccacacatac

SEQ ID NO: 21 - DNA, 29 bp, Artificial Sequence
  chemically treated genomic DNA (Homo sapiens)
ccatccccaaaaacacaaaccacacatac

SEQ ID NO: 22 - DNA, 27 bp, Artificial Sequence
  chemically treated genomic DNA (Homo sapiens)
catccccaaaaacacaaaccacacata

SEQ ID NO: 23 - DNA, 26 bp, Artificial Sequence
  chemically treated genomic DNA (Homo sapiens)
atccccaaaaacacaaaccacacata

SEQ ID NO: 24 - DNA, 28 bp, Artificial Sequence
  chemically treated genomic DNA (Homo sapiens)
ccatccccaaaaacacaaaccacacata

SEQ ID NO: 25 - DNA, 29 bp, Artificial Sequence
  chemically treated genomic DNA (Homo sapiens)
cccatccccaaaaacacaaaccacacata

SEQ ID NO: 26 - DNA, 28 bp, Artificial Sequence
  chemically treated genomic DNA (Homo sapiens)
cccatccccaaaaacacaaaccacacat

SEQ ID NO: 27 - DNA, 24 bp, Artificial Sequence
  chemically treated genomic DNA (Homo sapiens)
ccgaaaacgcgaaccgcgcgtact

SEQ ID NO: 28 - DNA, 32 bp, Artificial Sequence
  chemically treated genomic DNA (Homo sapiens)
cactaataacgaaaactacgacgacgaaactt

SEQ ID NO: 29 - DNA, 22 bp, Artificial Sequence
  chemically treated genomic DNA (Homo sapiens)
aaaacgcgaaccgcgcgtactc

SEQ ID NO: 30 - DNA, 25 bp, Artificial Sequence
  chemically treated genomic DNA (Homo sapiens)
aaccgcgcgtactcactaataacga

SEQ ID NO: 31 - DNA, 32 bp, Artificial Sequence
  chemically treated genomic DNA (Homo sapiens)
tcactaataacgaaaactacgacgacgaaact

SEQ ID NO: 32 - DNA, 35 bp, Artificial Sequence
  chemically treated genomic DNA (Homo sapiens)
cgcgtactcactaataacgaaaactacgacgacga

SEQ ID NO: 33 - DNA, 33 bp, Artificial Sequence
  chemically treated genomic DNA (Homo sapiens)
tggagtttcgtcgtcgtagttttcgttattagt

SEQ ID NO: 34 - DNA, 2501 bp, Artificial Sequence
  chemically treated genomic DNA (Homo sapiens)
gattttagttatagttttttaaggtttagtattttttttttttgttcgggtatggttatttacgtaggaggttttgagtgagtttttttgttacgtttttacggttattattttttttttttagttttggttttgatttgttagtagtatgcgtagggtcgcgtagcggtttgcggggagggagaagtacgagatgtggggatcgggtcgatttcgtttcgtagtaattcggggaggggttaggagtgtagggagggaatagggaaataggttttttcgaagattttatataatattggggcggggagtaggtatggcgggagaggcggggaataggaaggaggttcggggtaaaagttatacgacggagggataagggggttcggattttttcgggtgggcgaggggttgtgggttgtagttttagtttttgttttttttttttgttagatatatgtttttatttcgaattgggaaatagattacggtgtagggcggtattgtagcgaataaagaaaagtttgttggagttcgggggaggatgttaaggcgcggtgagcgtagtttgtttttttttttcgttttcggggttttattttttttcgaggcgtttcgggttttttgaaagtcgttaacggtattggggacgttttgggttttttaggttttcgtttcgggtttcgaggtgggcgaggagttttgtcgggagttcgggtttgatgttgcgggttggttttatgttgggagttttgagttttattttcggggacgcgggtcgcgcgtatttattggtggcgaagattgcggcggcgaaattttagcgaaggtttcgcggttttcgagttttataagggtggtttcgtttcgtttcgttttagtgttgagttacggcgtcggtcgtttttttggagggtttcgcggattttcgtcggttttagtttcggcggtcgttgtatttcgggcgtcggtcgtagaggggcgttttggagttttcggagtcgtcgcgtagttggtcggggaagttttttttttttttttaggtttttagcggggtttagggagtaaatagatagtaggaagaggatcgtagcgaagtgtgcgtagcgaattggcgcgtcgggatatcgcggggggaaattttttaagatcgttgcgatttcggagtttgtatattcgttttatagggtaggggagaggggtggaggtcgtttagaggaaaggaaattgttttattttattttattttattttatttttttattttattttattttattttattttattttattttattttattttattttattttgtgttattttattttattttatgacgtagttttacgttgtggtttaggttggagtgtagtggcgcgatttcggcggtttattgtaattttcgtttttcgggtttaagtaattttgttttagtttttcgagtaggtggaattataggtgcgtgttatatttggttgatttttgtatttttagtagagacggggttttattatgttggtcgggttggtttcgaatttttgattttaggtgatttgtacgtttcggttttttaaagtgttgggattataggcgtgagttattacgtttggtcgtttaatttttatttgaagttttggggtatatgtagaggatgtgtaggtttgttatataggtgtgtgcgttatgatggtttgttgtatagattattttattatttaggtattaagtttagtattttttagttattttttttggtattttttttttttagtatttcgtttaataggtattagtgtgtgttgatcgtcgttatgtgattatgtgtttttattgtttagtttttatttataagtgagattatgcggtttcgttggttttttgtttttgtgtgagtttgttgaggttaacggtttttagttttatttatgtttttgtaaaggatatgattacgttttttttagtggttgtgttttaggttattttttttggttttgttgtttattttttgttgatttgtagatttttatttattttagatattgattttttgttggttttagatatgatagatagttttttttattttattaattgttaagtttgtttaaggagttttttatgaaataaaattcgttaatttaagtgtaattaaatttagtaagggatttttgtggtggggaagaggttggtgtttatgttgtatttttaaaattttatttaatgtagttattaaaaagaattagattatgttttttgtgggaatatggatggagttagaggttattatttttagtaaattaatgtaggaatagaaatttaaatattggatgtttttatttgtaagtgggagttaaatgatgagaatttataatataaataaggaaataatagatattgtggttgattttagggtgtaggatgggaggaaggagaggagtagaaaagagaattattgggtattcggtataatatttgggtgatgaaatattttgtataataa

SEQ ID NO: 35 - DNA, 22 bp, Artificial Sequence
  chemically treated genomic DNA (Homo sapiens)
gggtttagagtttttagtatgg

SEQ ID NO: 36 - DNA, 23 bp, Artificial Sequence
  chemically treated genomic DNA (Homo sapiens)
tactaaaaactctaaaccccatc

SEQ ID NO: 37 - DNA, 23 bp, Artificial Sequence
  chemically treated genomic DNA (Homo sapiens)
gatggggtttagagtttttagta

SEQ ID NO: 38 - DNA, 23 bp, Artificial Sequence
  chemically treated genomic DNA (Homo sapiens)
tactcactaataacraaaactac

SEQ ID NO: 39 - DNA, 23 bp, Artificial Sequence
  chemically treated genomic DNA (Homo sapiens)
gtagttttcgttattagtgagta

SEQ ID NO: 40 - DNA, 17 bp, Artificial Sequence
  chemically treated genomic DNA (Homo sapiens)
ctctaaaccccatcccc

SEQ ID NO: 41 - DNA, 17 bp, Artificial Sequence
  chemically treated genomic DNA (Homo sapiens)
ggggatggggtttagag

SEQ ID NO: 42 - DNA, 24 bp, Artificial Sequence
  chemically treated genomic DNA (Homo sapiens)
gttgggagttttgagttttatttt

SEQ ID NO: 43 - DNA, 19 bp, Artificial Sequence
  R
aaaccttcrctaaaatttc

SEQ ID NO: 44 - DNA, 19 bp, Artificial Sequence
  chemically treated genomic DNA (Homo sapiens)
gaaattttagcgaaggttt

SEQ ID NO: 45 - DNA, 25 bp, Artificial Sequence
  chemically treated genomic DNA (Homo sapiens)
cgcggttcgcgttttcggggatggg

SEQ ID NO: 46 - DNA, 28 bp, Artificial Sequence
  chemically treated genomic DNA (Homo sapiens)
cccatccccaaaaacacaaaccacacat

SEQ ID NO: 47 - DNA, 28 bp, Artificial Sequence
  chemically treated genomic DNA (Homo sapiens)
atgcgcggttcgcgttttcggggatggg

SEQ ID NO: 48 - DNA, 34 bp, Artificial Sequence
  chemically treated genomic DNA (Homo sapiens)
ctaataacaaaaactacaacaacaaaactccaac

SEQ ID NO: 49 - DNA, 34 bp, Artificial Sequence
  chemically treated genomic DNA (Homo sapiens)
gttggagtttcgtcgtcgtagttttcgttattag

SEQ ID NO: 50 - DNA, 24 bp, Artificial Sequence
  chemically treated genomic DNA (Homo sapiens)
cccatccccaaaaacacaaaccac

SEQ ID NO: 51 - DNA, 24 bp, Artificial Sequence
  chemically treated genomic DNA (Homo sapiens)
gcggttcgcgttttcggggatggg

SEQ ID NO: 52 - DNA, 30 bp, Artificial Sequence
  chemically treated genomic DNA (Homo sapiens)
ctaaaatttcaccaccacaatcttcaccac

SEQ ID NO: 53 - DNA, 30 bp, Artificial Sequence
  chemically treated genomic DNA (Homo sapiens)
gtggcgaagattgcggcggcgaaattttag

SEQ ID NO: 54 - DNA, 20 bp, Artificial Sequence
  chemically treated genomic DNA (Homo sapiens)
ggttttagggaatttttttt

SEQ ID NO: 55 - DNA, 20 bp, Artificial Sequence
  chemically treated genomic DNA (Homo sapiens)
ggttttagggaatttttttt

SEQ ID NO: 56 - DNA, 17 bp, Artificial Sequence
  chemically treated genomic DNA (Homo sapiens)
ctttcccaaatccccaa

SEQ ID NO: 57 - DNA, 17 bp, Artificial Sequence
  chemically treated genomic DNA (Homo sapiens)
ttggggatttgggaaag

SEQ ID NO: 58 - DNA, 19 bp, Artificial Sequence
  chemically treated genomic DNA (Homo sapiens)
gaaaggggaaaggtttttt

SEQ ID NO: 59 - DNA, 19 bp, Artificial Sequence
  chemically treated genomic DNA (Homo sapiens)
gaaaggggaaaggtttttt

SEQ ID NO: 60 - DNA, 18 bp, Artificial Sequence
  chemically treated genomic DNA (Homo sapiens)
crccccaatactaaatca

SEQ ID NO: 61 - DNA, 18 bp, Artificial Sequence
  chemically treated genomic DNA (Homo sapiens)
tgatttagtattggggcg

SEQ ID NO: 62 - DNA, 22 bp, Artificial Sequence
  chemically treated genomic DNA (Homo sapiens)
gggaaagagggaaaggtttttt

SEQ ID NO: 63 - DNA, 22 bp, Artificial Sequence
  chemically treated genomic DNA (Homo sapiens)
gggaaagagggaaaggtttttt

SEQ ID NO: 64 - DNA, 22 bp, Artificial Sequence
  chemically treated genomic DNA (Homo sapiens)
ctccrccccaatactaaatcac

SEQ ID NO: 65 - DNA, 22 bp, Artificial Sequence
  chemically treated genomic DNA (Homo sapiens)
gtgatttagtattggggcggag

SEQ ID NO: 66 - DNA, 19 bp, Artificial Sequence
  Y
gatttyggggattttaggg

SEQ ID NO: 67 - DNA, 19 bp, Artificial Sequence
  chemically treated genomic DNA (Homo sapiens)
gatttcggggattttaggg

SEQ ID NO: 68 - DNA, 17 bp, Artificial Sequence
  chemically treated genomic DNA (Homo sapiens)
ccccaatactaaatcac

SEQ ID NO: 69 - DNA, 17 bp, Artificial Sequence
  chemically treated genomic DNA (Homo sapiens)
gtgatttagtattgggg

SEQ ID NO: 70 - DNA, 20 bp, Artificial Sequence
  chemically treated genomic DNA (Homo sapiens)
ttttagagatgtttaggagc

SEQ ID NO: 71 - DNA, 20 bp, Artificial Sequence
  chemically treated genomic DNA (Homo sapiens)
ttttcgcgatgtttcggcgc

SEQ ID NO: 72 - DNA, 19 bp, Artificial Sequence
  chemically treated genomic DNA (Homo sapiens)
atcacaacaccaaccacac

SEQ ID NO: 73 - DNA, 19 bp, Artificial Sequence
  chemically treated genomic DNA (Homo sapiens)
gagcggtcggcgtcgtgat

SEQ ID NO: 74 - DNA, 28 bp, Artificial Sequence
  chemically treated genomic DNA (Homo sapiens)
ccccaatactaaatcacaacaccaacca

SEQ ID NO: 75 - DNA, 28 bp, Artificial Sequence
  chemically treated genomic DNA (Homo sapiens)
cggtcggcgtcgtgatttagtattgggg

SEQ ID NO: 76 - DNA, 29 bp, Artificial Sequence
  chemically treated genomic DNA (Homo sapiens)
atactaaatcacaacaccaaccactcttc

SEQ ID NO: 77 - DNA, 29 bp, Artificial Sequence
  chemically treated genomic DNA (Homo sapiens)
gaagagcggtcggcgtcgtgatttagtat

SEQ ID NO: 78 - DNA, 28 bp, Homo Sapiens
gagtttcgccgccgcagtcttcgccacc

SEQ ID NO: 79 - DNA, 28 bp, Artificial Sequence
  chemically treated genomic DNA (Homo sapiens)
gagtttcgtcgtcgtagttttcgttatt

SEQ ID NO: 80 - DNA, 24 bp, Artificial Sequence
  1000.10B22
cccatccccaaaaacacaaaccac

SEQ ID NO: 81 - DNA, 24 bp, Artificial Sequence
  1000.10B23
cccatccccaaaaacacaaaccgc

SEQ ID NO: 82 - DNA, 24 bp, Artificial Sequence
  1000.10B24
cccatccccaaaaacacgaaccac

SEQ ID NO: 83 - DNA, 24 bp, Artificial Sequence
  1000.10B25
cccatccccaaaaacgcaaaccac

SEQ ID NO: 84 - DNA, 24 bp, Artificial Sequence
  1000.10B26
cccatccccgaaaacacaaaccac

SEQ ID NO: 85 - DNA, 24 bp, Artificial Sequence
  1000.B26.2
cccatcccccaaaacacaaaccac

SEQ ID NO: 86 - DNA, 24 bp, Artificial Sequence
  1000.B26.3
cccatcccctaaaacacaaaccac

SEQ ID NO: 87 - DNA, 24 bp, Artificial Sequence
  1000.10B27
cccatccccgaaaacgcaaaccac

SEQ ID NO: 88 - DNA, 24 bp, Artificial Sequence
  1000.10B28
cccatccccgaaaacacgaaccac

SEQ ID NO: 89 - DNA, 24 bp, Artificial Sequence
  1000.10B29
cccatccccgaaaacacaaaccgc

SEQ ID NO: 90 - DNA, 24 bp, Artificial Sequence
  1000.10B30
cccatccccaaaaacgcgaaccac

SEQ ID NO: 91 - DNA, 24 bp, Artificial Sequence
  1000.10B31
cccatccccaaaaacgcaaaccgc

SEQ ID NO: 92 - DNA, 24 bp, Artificial Sequence
  1000.10B32
cccatccccaaaaacacgaaccgc

SEQ ID NO: 93 - DNA, 28 bp, Artificial Sequence
  1000.10B100
catccccaaaaacacaaaccacacatac

SEQ ID NO: 94 - DNA, 27 bp, Artificial Sequence
  1000.10B101
atccccaaaaacacaaaccacacatac

SEQ ID NO: 95 - DNA, 29 bp, Artificial Sequence
  1000.10B102
ccatccccaaaaacacaaaccacacatac

SEQ ID NO: 96 - DNA, 27 bp, Artificial Sequence
  1000.10B103
catccccaaaaacacaaaccacacata

SEQ ID NO: 97 - DNA, 28 bp, Artificial Sequence
  1000.10B105
ccatccccaaaaacacaaaccacacata

SEQ ID NO: 98 - DNA, 29 bp, Artificial Sequence
  1000.10B106
cccatccccaaaaacacaaaccacacata

SEQ ID NO: 99 - DNA, 28 bp, Artificial Sequence
  1000.10B107
cccatccccaaaaacacaaaccacacat

SEQ ID NO: 100 - DNA, 27 bp, Artificial Sequence
  1000.10B107-G
cccatccccaaaaaacaaaccacacat

SEQ ID NO: 101 - DNA, 33 bp, Artificial Sequence
  1000.10B117.2
cccatcccctaaaacactaaccacacatactca

SEQ ID NO: 102 - DNA, 33 bp, Artificial Sequence
  1000.10B118.2
cccatcccctaaaacacaaacctcacatactca

SEQ ID NO: 103 - DNA, 32 bp, Artificial Sequence
  1000.10B119
aaaccccatcccctaaaacactaaccacacat

SEQ ID NO: 104 - DNA, 32 bp, Artificial Sequence
  1000.10B120
aaaccccatcccctaaaacacaaacctcacat

SEQ ID NO: 105 - DNA, 24 bp, Artificial Sequence
  1000.10-fluo2
tgaggtttttgttggagttttgtt

SEQ ID NO: 106 - DNA, 30 bp, Artificial Sequence
  1000.10-red2
tgtagtttttgttattagtgagtatgtgtg

SEQ ID NO: 107 - DNA, 19 bp, Artificial Sequence
  1000.10-fluo5
gttggagtttcgtcgtcgt

SEQ ID NO: 108 - DNA, 21 bp, Artificial Sequence
  1000.10-fluo10
ttcgtcgtcatagttttcgtt

SEQ ID NO: 109 - DNA, 21 bp, Artificial Sequence
  1000.10-fluo11
ttcgtcatcatagttttcgtt

SEQ ID NO: 110 - DNA, 24 bp, Artificial Sequence
  1000.10-fluo12
agtttcgtcgtcatagttttcgtt

SEQ ID NO: 111 - DNA, 24 bp, Artificial Sequence
  1000.10-fluo20
agtttcgtcgtcgtagttttcgtt

SEQ ID NO: 112 - DNA, 21 bp, Artificial Sequence
  1000.10-fluo1SNP
ttcgttatcgtagttttcgtt

SEQ ID NO: 113 - DNA, 27 bp, Artificial Sequence
  1000.10-fluoSNP2
tggagtttcgttatcgtagttttcgtt

SEQ ID NO: 114 - DNA, 24 bp, Artificial Sequence
  1000.10-red5
gttttcgttattagtgagtacgcg

SEQ ID NO: 115 - DNA, 18 bp, Artificial Sequence
  1000.10-red6 Y
tagtgagtacgcgcggtt

SEQ ID NO: 116 - DNA, 18 bp, Artificial Sequence
  1000.10-red7
tagtgagtacgtgcggtt

SEQ ID NO: 117 - DNA, 20 bp, Artificial Sequence
  1000.10-red20
tagtgagtacgcgcggttcg

SEQ ID NO: 118 - DNA, 19 bp, Artificial Sequence
  HM4 Probe fluo
cgtcgtcgtagttttcgtt

SEQ ID NO: 119 - DNA, 21 bp, Artificial Sequence
  1000.12-fluo
cttcgccaccaataaatacgc

SEQ ID NO: 120 - DNA, 13 bp, Artificial Sequence
  1000.12-red
cgacccgcgtccc

SEQ ID NO: 121 - DNA, 2501 bp, Artificial Sequence
  chemically treated genomic DNA (Homo sapiens)
ttgttgtatagaatattttattatttaggtattatgtcgagtatttaatagtttttttttttgtttttttttttttttttattttgtattttggagttaattatagtgtttgttgtttttttgtttgtgttataagtttttattatttagtttttatttataagtgagaatatttagtatttggatttttgtttttgtattagtttgttaaggataatagtttttagttttatttatgtttttataaaagatatgatttagttttttttaatggttgtattaaatgaagttttaaagatataatataaatattaattttttttttattataaaaattttttgttgaatttgattatatttaaattaacgagttttgttttatgaaagattttttggataaatttgatagttgatggaataggagaagttgtttgttatgtttaaagttaataagagattaatatttagaataaatggagatttgtaaattaatagaaagtaggtagtaaagttaaagaaaatagtttaaggtatagttattaaaaggaacgtgattatgttttttgtagggatatgggtggagttggaagtcgttagttttagtaaatttatataggaatagaaaattagcgagatcgtatggttttatttataagtgggagttgaataatgagaatatatggttatatggcggcgattaatatatattggtgtttgttgagcggggtgttggggagggagagtattaggaagaatagttaagggatattgggtttaatatttgggtgatgggatgatttgtatagtaaattattatggcgtatatatttatgtaataaatttgtatattttttatatgtattttagaattttaaataaaagttggacggttaggcgtggtggtttacgtttgtaattttagtattttgggaagtcgaggcgtgtagattatttaaggttaggagttcgagattagttcggttaatatggtgaaatttcgtttttattaaaaatataaaaattagttagatgtggtacgtatttataattttatttattcgggaggttgaagtagaattgtttgaattcgagaggcggaggttgtagtgagtcgtcgagatcgcgttattgtattttagtttgggttatagcgtgagattacgttataaaataaaataaaataatataaaataaaataaaataaaataaaataaaataaaataaaataaaataaaataaaataaaaaaataaaataaaataaaataaaataaagtaatttttttttttttaagcggtttttattttttttttttgttttgtgaagcgggtgtgtaagtttcgggatcgtagcggttttagggaattttttttcgcgatgtttcggcgcgttagttcgttgcgtatatttcgttgcggttttttttttgttgtttgtttattttttaggtttcgttggggatttgggaaagagggaaaggttttttcggttagttgcgcggcgatttcggggattttagggcgtttttttgcggtcgacgttcggggtgtagcggtcgtcggggttggggtcggcgggagttcgcgggattttttagaagagcggtcggcgtcgtgatttagtattggggcggagcggggcgggattatttttataaggttcggaggtcgcgaggttttcgttggagtttcgtcgtcgtagttttcgttattagtgagtacgcgcggttcgcgttttcggggatggggtttagagtttttagtatggggttaattcgtagtattaggttcgggttttcggtagggtttttcgtttatttcgagattcgggacgggggtttaggggatttaggacgtttttagtgtcgttagcggtttttagggggttcggagcgtttcggggagggatgggatttcgggggcggggagggggggtagattgcgtttatcgcgttttggtattttttttcgggttttagtaaatttttttttgttcgttgtagtgtcgttttatatcgtggtttattttttagttcgaggtaggagtatgtgtttggtagggaagggaggtaggggttggggttgtagtttatagtttttcgtttattcggagagattcgaatttttttattttttcgtcgtgtggtttttatttcgggttttttttttgtttttcgtttttttcgttatgtttgtttttcgttttagtgttgtgtgaaattttcggaggaatttgtttttttgttttttttttgtatttttgattttttttcgggttgttgcgaggcggagtcggttcggtttttatatttcgtattttttttttttcgtaggtcgttgcgcggttttgcgtatgttgttggtagattagggttagagttggaaggaggaggtggtgatcgtggagacgtggtaggagggtttatttaaagttttttgcgtaagtgattatgttcgggtaaggggagggggtgttgggttttagggggttgtgattaggatt

(19) United States
(12) Patent Application Publication
(10) Pub. No.: US 2007/0117093 A1
(43) Pub. Date: May 24, 2007

(54) Heavymethyl assay for the methylation analysis of the gstpi gene

(76) Inventors: Reimo Tetzner, Berlin (DE); Jurgen Distler, Berlin (DE)

Correspondence Address: KRIEGSMAN & KRIEGSMAN, 30 TURNPIKE ROAD, SUITE 9, SOUTHBOROUGH, MA, 01772, US

(21) Appl. No.: 10/562,196

(22) PCT Filed: Jun. 24, 2004
(86) PCT No.: PCT/EP04/06843

§ 371 Date: Dec. 23, 2005

(30) Foreign Application Priority Data
Jun. 24, 2003 (EP) 03090189.6

(51) Int. Cl. C12Q 1/68

(52) U.S. Cl. 435/6

(57) ABSTRACT

Described herein is a method for the detection of cyto-sine methylation in DNA samples, wherein the following steps are conducted: a genomic DNA sample, which comprises the DNA to be investigated as well as background DNA is treated with bisulfite (&equals; disulfite, hydrogen sulfite) in such a way that all of the unmethylated cytosine bases are converted to uracil, while the 5-methylcytosine bases remain unchanged; the bisulfite treated DNA sample is amplified with the use of at least 2 primer oligonucleotides as well as a polymerase, wherein the DNA to be investigated is pre-ferred over the background DNA as the template, and a control fragment is amplified simultaneously to the am-plification of the bisulfite treated DNA within the same reaction mixture the amplified products are analyzed and the methylation status in the DNA to be investigated is concluded from the presence and /or the amount of the amplified products and/or from the analysis of additional positions.

### BACKGROUND

Correlation of aberrant DNA methylation with cancer. Aberrant DNA methylation within CpG ‘islands’ is characterized by hyper- or hypomethylation of CpG dinucleotide sequences leading to abrogation or over-expression of a broad spectrum of genes, and is among the earliest and most common alterations found in, and correlated with human malignancies. Additionally, abnormal methylation has been shown to occur in CpG-rich regulatory elements in intronic and coding parts of genes for certain tumors. In colon cancer, aberrant DNA methylation constitutes one of the most prominent alterations and inactivates many tumor suppressor genes.

Aside from the specific hypermethylation of tumor suppressor genes, an overall hypomethylation of DNA can be observed in tumor cells. This decrease in global methylation can be detected early, far before the development of frank tumor formation. A correlation between hypomethylation and increased gene expression has been determined for many oncogenes.

Prostate cancer. The prostate is a male sex accessory gland, comprising about 30 to 50 branched glands. It is surrounded by a fibroelastic capsule that separates the gland into discrete lobes. Benign prostate hypertrophy is present in about 50% of men aged 50 or above, and in 95% of men aged 75 or above. Prostate cancer is a significant health care problem in Western countries with an incidence of 180 per 100,000 in the United States in 1999 (Cancer J. Clin., 49:8, 1999).

Diagnosis and prognosis of prostate cancer; deficiencies of prior art approaches. Different screening strategies have been employed with at least some degree of success to improve early detection of prostate cancer, including determination of levels of prostate specific antigen (“PSA”) and digital rectal examination. If a prostate carcinoma is suspected in a patient, diagnosis of cancer is confirmed or excluded by the histological and cytological analysis of biopsy samples for features associated with malignant transformation.

Prostate specific antigen levels of over 15 ng/ml are considered as indicative of prostate cancer and grounds for a biopsy. The biopsy, in turn, is used for histological and cytological analysis.

However, using routine histological examination, it is often difficult to distinguish benign hyperplasia of the prostate from early stages of prostate carcinoma, even if an adequate biopsy is obtained (McNeal J. E. et al., Hum. Pathol. 2001, 32:441-6). Furthermore, small or otherwise insufficient biopsy samples often impede the analysis.

Molecular markers would offer the advantage that they could be used to efficiently analyze even very small tissue samples, and samples whose tissue architecture has not been maintained. Within the last decade, numerous genes have been studied with respect to differential expression among benign hyperplasia of the prostate and different grades of prostate cancer.

Alternatively, high-dimensional mRNA based approaches may, in particular instances, provide a means to distinguish between different tumor types and benign and malignant lesions. However, application of such approaches as a routine diagnostic tool in a clinical environment is impeded and substantially limited by the extreme instability of mRNA, the rapidly occurring expression changes following certain triggers (e.g., sample collection), and, most importantly, by the large amount of mRNA needed for analysis which often cannot be obtained from a routine biopsy (see, e.g., Lipshutz, R. J. et al., Nature Genetics 21:20-24, 1999; Bowtell, D. D. L. Nature Genetics Suppl. 21:25-32, 1999).

The GSTP1 gene. The core promoter region of the Gluthione S-Transferase P gene (GSTP1; accession no. NM_{—}000852) has been shown to be hypermethylated in prostate tumor tissue. The glutathione S-transferase pi enzyme is involved in the detoxification of electrophilic carcinogens, and impaired or decreased levels of enzymatic activity (GSTPi impairment) have been associated with the development of neoplasms, particularly in the prostate. Mechanisms of GSTPi impairment include mutation (the GSTP*B allele has been associated with a higher risk of cancer) and methylation.

Prior art GSTP1 studies. Expression levels of the GSTP1 gene have been measured comparatively in high grade prostatic intraepithelial neoplasia of the transitional and peripheral zones by means of immunohistological staining (Bartels et al., Mol Pathol., 53:122-8, 2000; “Expression of pi-class glutathione S-transferase: two populations of high grade prostatic intraepithelial neoplasia with different relations to carcinoma”).

Lee et al., in U.S. Pat. No. 5,552,277, disclosed that the expression of the gluthione-S-transferase (GSI) Pi gene was downregulated in a significant proportion of prostate carcinomas. Moreover, by means of restriction enzyme analysis they were able to show that the promoter region of the GSTPi gene was upmethylated (hypermethylated) in prostate carcinomas as opposed to normal prostate and leukocyte tissue. However, due to the limited and imprecise nature of the analysis technique used (HpaIII digestion, followed by Southern blotting) the exact number and position of the methylated CG dinucleotides were not characterized.

Douglas et al. (WO9955905) used a method comprising bisulfite treatment, followed by methylation specific PCR to show that prostate carcinoma-specific GSTPi hypermethylation was localized to the core promoter regions, and localized a number of CpG positions that had not been characterized by Lee et al.

Herman and Baylin (U.S. Pat. No. 6,017,704) describe the use of methylation specific primers for methylation analysis, and describe a particular primer pair suitable for the analysis of the corresponding methylated GSTPi promoter sequence. This methylation detection assay is called MSP. The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146.

In the PCT application document WO 02/072880 an assay is described, which uses methylation sensitive blocldng oligos, that distinguish between cytosines, which have been methylated prior to the so called bisulfite-treatment and cytosines, which have been unmethylated prior to said treatment, in combination with common PCR primers. As a result only those sequences will be amplified that show a methylation pattern which is not recognized by said blocldng oligos. This assay is referred to as the HeavyMethyl assay, which can also be performed as a real time PCR, with the use of probes like for example a Taqman probe of LightCycler probes or similar.

The term “HeavyMethyl” assay, as used in the description of the invention, refers to a HeavyMethyl MethylLight™ assay, which is a variation of the MethylLight™ assay, wherein the MethyLight™ assay is combined with methylation specific blocking probes covering CpG positions between the amplification primers.

However, with respect to the use of methylation detection assays of markers as for example the GSTPi markers, the prior art is limited with respect to the sensitivity that can be achieved for CpG methylation results obtained for GSTPi promoter CpG sequences, that have been characterized as showing differential methylation status.

There are no disclosures, suggestions or teachings in the prior art as to how to detect the methylation status of a relevant CpG site within the GSTP1 gene or its regulatory regions with the sensitivity required for a routine testing of bodily fluids like, for example, serum, plasma, lymph, or sperm, but especially urine, where the nucleic acid levels are especially low.

Furthermore, there are no disclosures, suggestions or teachings in the prior art of how to improve the so called HeavyMethyl assay in itself, to make its result more reliable and robust.

### DESCRIPTION OF THE INVENTION

In the PCT application document WO 02/072880 and in a publication by Cottrell et al. (Cottrell SE et al. Nucleic Acids Res. 2004 Jan. 13;32(1):e10) a methylation specific amplification assay is described, which uses methylation sensitive blocking oligos, that distinguish between cytosines, which have been methylated prior to “bisulfite-treatment” as described in Olek et a. (Nucleic Acids Res. (1996) 24, 5064-5066) and cytosines, which have been unmethylated prior to said treatment, in combination with common methylation unspecific PCR primers. As a result only those sequences will be amplified that show a methylation pattern which is not recognized by said blocking oligos. This assay is referred to as the HeavyMethyl assay, which can also be performed as a real time PCR, with the use of probes (also referred to as detection probes) like for example a Taqman probe or LightCycler probes or similar.

The term “HeavyMethyl” assay, as used in the description of this invention, often also refers to a HeavyMethyl MethyLight™ assay, which is a variation of the MethyLight™ assay, wherein the MethyLight™ assay is combined with methylation specific blocking probes covering CpG positions between the amplification primers.

A specific “HeavyMethyl assay” according to this invention shall be sufficiently defined by the combination of primers and blocker(s) used.

The selection of the ideal probe is another way to improve the performance of an assay, however it is regarded as crucial for the performance of the assays, that the right combination of primers and probes is employed. In this application specific primers and blockers as well as specific combinations are disclosed to allow the improvement of a diagnostic method based on the methylation analysis of the gene GSTP1 and its regulatory regions, especially when using the HeavyMethyl method as the method of choice.

The detection of cytosine methylation by means of a real-time PCR has become known as “MethyLight”. This method, allowing to detect the methylation status of individual positions or a few positions directly in the course of a PCR, so that a subsequent analysis of the products is spared, is described in U.S. Pat. No. 6,331,393 to Laird et al. (WO 00/70090).

Herein disclosed are means of how to improve the HeavyMethyl assay performance specifically for the gene GSTP1 and its regulatory region, and more specifically the genomic regions referred to as promoter region or region suitable for analyzing exon 1. The according “artificial” sequence representing a bisulfite converted sequence of the methylated variant of said genomic region is specified in SEQ ID NO 33 (sense) and SEQ ID NO 34 (antisense).

The method according to the invention enables the sensitive detection of the methylation status of specific CpG positions in the GSTP1 genomic region as given in SEQ ID XXXX. Disclosed are the means on how to detect the methylation of specific CpG sites within the nucleic acid defined by the sequence nt 1183 to nt 1309 of Genbank Accession number M24485.1 or also nt 1845 to nt 1624 of Genbank accession number AY324387.

It is an especially preferred embodiment to detect the methylation status of CpG sites characterized as being located in the genomic region referred to as GSTP1 exon 1 (SEQ ID NO 78) with the method referred to as HeavyMethyl as described in detail in WO 02/072880.

Several embodiments of this invention are specified “assay formats”. Provided are specific primer sequences and blocker-oligo sequences and the specific combinations of primer pairs and the according blockers. These specific combinations are surprisingly more effective than other combinations and are therefore special as compared to the general principle, known in the art.

The method according to the invention is therefore characterized as performing a HeavyMethyl assay and employing thereby at least one primer (forward) out of the group consisting of SEQ ID NOs: 2, 42, 54, 58, 62 and 66 and one primer (reverse) out of the group consisting of SEQ ID NOs: 3, 36, 38, 40, 43, 56, 60, 64 and 68 and at least one blocker out of the group of SEQ ID NO 4, 46, 48, 50, 52, 70, 72, 74, 76, 80, 81, 82, 83, 84, 85, 86, 87, 88, 89, 90, 91, 92, 93, 94, 95, 96, 97, 98, 99, 100, 102, 103 and SEQ ID NO:104.

It is preferred that at least one of the blocking oligo nucleotides (also referred to as the additional oligonucleotides or blockers) is taken out of the group consisting of SEQ ID NO 4, 46, 48, 50, 52, 70, 72, 74, and SEQ ID NO 76. These are the oligo nucleotides which are essential embodiments of the specific assays described herein. It is especially preferred that according to the invention at least one of the additional oligonucleotides used in the assay referred to as HeavyMethyl, applied to analyse the GSTP1 gene is taken from the group consisting of SEQ ID NO 4, 46, 48, 50 and SEQ ID NO 52. These are the preferred blockers which inhibit the amplification of bisulfite converted unmethylated nucleic acids and therefore allow the specific amplification of bisulfite converted methylated nucleic acids comprising a sequence that is at least in part identical to the SEQ ID NO 79 (the bisulfite converted exon 1).

It is especially preferred that one of the combinations as described in tables 1 to 4 is used. It is therefore a preferred embodiment of the invention that an assay out of the group consisting of assays Exon HM 1, Exon HM 2, Exon HM3, Exon HM 4, Exon HM 5, P HM 1, P HM 2, P HM3 and P HM 4 is performed. It is especially preferred that an assay out of the group consisting of assays Exon HM 1, Exon HM 2, Exon HM3, Exon HM 4 and Exon HM 5 is performed, because these assays focus on the analysis of the region following the promoter region of GSTP1, and are especially suitable to analyze the methylation status of CpG positions in SEQ ID 78 (exon 1), which are informative in view of the diagnosis of an individual who the DNA sample was derived from. The assays are characterized further in tables 1 and 2. It is also preferred that an assay out of the group of assays P HM 1, P HM 2, P HM 3 and P HM 4 is performed. The assays P HM 1, P HM 2, P HM3 and P HM 4 are especially preferred when the promoter region of GSTP1 is to be investigated and analyzed. These assays are characterized in tables 3 and 4.

The following assays are preferred embodiments of the invention. The preferred combinations of primers which generate the fragments of interest and preferred blockers are listed in the tables 1 to 4 as specified below. The most preferred combinations (specific assay formats) are listed in the following 2 tables 1 and 2.

TABLE 1

List of preferred primer combinations (fragments) suitable
for the analysis of CpG methylation within the region of
exon 1 of GSTP1 with a HM assay. In a) assays are listed
that work on the bisulfite converted sense strand (bisu 1)
and in b) is the assay listed that works on the bisulfite
converted anti-sense strand (bisu 2):

| | | | |
|---|---|---|---|
| Refer- | | | |
| ence | Forward Primer, | Reverse Primer, | |
| Number | SEQ ID NO; | SEQ ID NO; | |
| Fragment | region within bisu 1, | region within bisu 1, | Fragment |
| number | SEQ ID NO | SEQ ID NO | size |

a)

| | | | |
|---|---|---|---|
| Ex HM 1 | GGGATTATTTTTATAAGGTT | CCATACTAAAAACTCTAAACCC | F1R4 |
| | SEQ ID NO 2 | SEQ ID NO 3 | |
| gstp1.10 GGGATTATTTTTATAAGGTT | GGGTTTAGAGTTTTTAGTATGG | 126 bp | |
| | SEQ ID NO 2 | SEQ ID NO 35 | |
| Ex HM 2 | GGGATTATTTTTATAAGGTT | TACTAAAAACTCTAAACCCCATC | F1R5 |
| | SEQ ID NO 2 | SEQ ID NO 36 | |
| gstp1.10 GGGATTATTTTTATAAGGTT | GATGGGGTTTAGAGTTTTTAGTA | 123 bp | |
| | SEQ ID NO 2 | SEQ ID NO 37 | |
| Ex HM 3 | GGGATTATTTTTATAAGGTT | TACTCACTAATAACKAAAACTAC | F1R6 |
| | SEQ ID NO 2 | SEQ ID NO 38 | |
| gstp1.14 GGGATTATTTTTATAAGGTT | GTAGTTTTCGTTATTAGTGAGTA | | |
| | SEQ ID NO 2 | SEQ ID NO 39 | |
| Ex.HM 4 | GGGATTATTTTTATAAGGTT | CTGTAAACCCCATCCCC | F1R8 |
| | SEQ ID NO 2 | SEQ ID NO 40 | |
| gstp1.10 | GGGATTATTTTTATAAGGTT | GGGGATGGGGTTTAGAG | |
| | SEQ ID NO 2 | SEQ ID NO 41 | |

| | | |
|---|---|---|
| | Forward Primer | Reverse Primer |
| | SEQ ID NO and | SEQ ID NO and |
| Reference | region within bisu 2 | region within bisu 2 |
| Number | SEQ ID NO | SEQ ID NO |

b)

| | | |
|---|---|---|
| Ex HM 5 | GTTGGGAGTTTTGAGTTTTATTTT | AAACCTTCKCTAAAATTTC |
| | SEQ ID NO 42 | SEQ ID NO 43 |
| gstp1.12 | GTTGGGAGTTTTGAGTTTTATTTT | GAAATTTTAGCGAAGGTTT |
| F1R2 | SEQ ID NO 42 | SEQ ID NO 44 |

The sequence of the primers are designated in 5′ to 3′ direction. The type of template amplified by the primer is specified as bisulfite treated DNA (bisulfite) generated from methylated genomic DNA or unmethylated genomic DNA.

Wherein a K is presented in the sequence it is indicating the use of a “universal base” as explained in the EUROGENTEC 2004 catalog (see www.eurogentec.com). Universal bases can be used instead of degenerated bases, but in the scope of the invention it is also allowed to use degenerated bases. The advantage of using universal bases is, that the hybridising probe is not diluted by the non-pairing components of the degeneracy. The universal base used in this context is characterized as a hybridising efficiently with pyrimidines, such as C or T.

TABLE 2

List of preferred blockers suitable for the
analysis of CpG methylation within the region
of exon 1 of GSTP1 with a HM assay according
to the fragments listed above:

| | Blocker for unmethylated | SEQ |
|---|---|---|
| Reference | and region within | ID |
| Number | up-methylated bisu 1 | NO |
| Ex HM 1 | CCCATCCCCaAAAACaCaAACCaCa; | 4 |
| gstp1.10 | CGCGGTTCGCGTTTTCGGGGATGGG | 45 |
| B20 | | |
| Ex HM 2 | CCCATCCCCaAAAACaCaAACCaCaCAT; | 46 |
| gstp1.10 | ATGCGCGGTTCGCGTTTTCGGGGATGGG | 47 |
| B107 | | |
| Ex HM 3 | CTAATAACAAAAACTACaACaACaAAACTCCAAC; | 48 |
| gstp1.14 | GTTGGAGTTTCGTCGTCGTAGTTTTCGTTATTAG | 49 |
| Ex HM 4 | CCCATCCCCaAAAACaCaAACCaC; | 50 |
| gstp1.10 | GCGGTTCGCGTTTTCGGGGATGGG | 51 |
| B22 | | |
| | Blocker for unmethylated | SEQ |
| Reference | sequence and region within | ID |
| Number | up-methylated bisu 2 | NO |
| Ex HM 5 | CTAAAATTTCaCCaCCaCAATCTTCaCCAC; | 52 |
| gstp1.12 | GTGGCGAAGATTGCGGCGGCGAAATTTTAG | 53 |

The sequence of the blockers are designated in 5′ to 3′ direction. Where an a (instead of A) is printed it indicates that the blocker nucleotide matches with a thymine that originated from an unmethylated cytosine. In the upmethylated bisulfite treated DNA these positions are shown as C, whereas they were printed as T in the sequence of the down-methylated DNA.

It is also possible to design HM assays for analysis of informative positions in the promoter region of GSTP1. Possible assay formats are listed in the following table.

TABLE 3

List of fragments suitable to detect CpG methylation
within the promoter region of GSTP1

| | Forward Primer (SEQ ID NO) | Reverse Primer (SEQ ID NO) |
|---|---|---|
| | and region within up- | and region within up- |
| Reference | methylated bisu 1 | methylated bisu 1 |
| Number | (SEQ ID NO) | (SEQ ID NO) |
| P HM 1 | GGTTTTAGGGAATTTTTTTT; | CTTTCCCAAATCCCCAA; |
| | SEQ ID NO 54 | SEQ ID NO 56 |
| | GGTTTTAGGGAATTTTTTTT | TTGGGGATTTGGGAAAG |
| | SEQ ID NO 55 | SEQ ID NO 57 |
| P HM 2 | GAAAGGGGAAAGGTTTTTT; | CKCCCCAATACTAAATCA; |
| | SEQ ID NO 58 | SEQ ID NO 60 |
| | GAAAGGGGAAAGGTTTTTT | TGATTTAGTATTGGGGCG |
| | SEQ ID NO 59 | SEQ ID NO 61 |
| P HM 3 | GGGAAAGAGGGAAAGGTTTTTT; | CTCCKCCCCAATACTAAATCAC; |
| | SEQ ID NO 62 | SEQ ID NO 64 |
| | GGGAAAGAGGGAAAGGTTTTTT | GTGATTTAGTATTGGGGCGGAG |
| | SEQ ID NO 63 | SEQ ID NO 65 |
| P HM 4 | GATTTYGGGGATTTTAGGG; | CCCCAATACTAAATCAC; |
| | SEQ ID NO 66 | SEQ ID NO 68 |
| | GATTTCGGGGATTTTAGGG | GTGATTTAGTATTGGGG |
| | SEQ ID NO 67 | SEQ ID NO 68 |

TABLE 4

List of blockers suitable for the analysis
of GpG methylation within the promoter
region of GSTP 1 with a HM assay according
to the fragments listed above:

| | Blocker for unmethylated; | SEQ |
|---|---|---|
| Reference | region within upmethylated | ID |
| Number | bisu 1 | NO |
| P HM 1 | TTTTaGaGATGTTTaGGaGC; | SEQ ID |
| | | NO 70 |
| | TTTTCGCGATGTTTCGGCGC | SEQ ID |
| | | NO 71 |
| P HM 2 | ATCACaACaCCaACCaCAC; | SEQ ID |
| | | NO 72 |
| | GAGCGGTCGGCGTCGTGAT | SEQ ID |
| | | NO 73 |
| P HM 3 | CCCCAATACTAAATCACaACaCCaACCa; | SEQ ID |
| | | NO 74 |
| | CGGTCGGCGTCGTGATTTAGTATTGGGG | SEQ ID |
| | | NO 75 |
| P HM 4 | ATACTAAATCACaACaCCaACCaCTCTTC; | SEQ ID |
| | | NO 76 |
| | GAAGAGCGGTCGGCGTCGTGATTTAGTAT | SEQ ID |
| | | NO 77 |

Preferred embodiments in this invention are specific and established assays. Especially those that are suited to analyse the methylation state of CpG positions within the region of exon 1 of the GSTP1 gene, which has the following (genomic) sequence of 28 bp (according to GenBank entry AY324387 it can be located at nt 1888 to nt 1915):

| | |
|---|---|
| GAGTTTCGCCGCCGCAGTCTTCGCCACC; | SEQ ID NO:78 |

which correlates to the following up-methylated bisulfite sequence:

| | |
|---|---|
| GAGTTTCGTCGTCGTAGTTTTCGTTATT | SEQ ID NO:79 |

Assays suitable to analyze these CpG sites will be referred to as Exon HM 1-Exon HM 5.
Exon HM 1:

In one of the preferred assays, assay Exon HM 1 a fragment of 126 bp (SEQ ID NO:1) is generated by the primer pair F1 (SEQ ID NO 2) and R4 (SEQ ID NO 3), which can be located at nt 1845-1970 of GenBank entry AY324387). Blockers that are suitable to be used to detect cytosine methylation within this fragment are listed in table in BBB. The performance of this assay is described in examples 1, 2, 3, 4 and 5.

Exon HM2:

In another one of the preferred assays, assay Exon HM 2 a fragment of 123 bp is generated by the primer pair F1 (SEQ ID NO 2) and R5 (SEQ ID NO 13 or SEQ ID NO 35), which can be located at nt 1845-1967 of GenBank entry AY324387).

Blockers that are suitable to be used to detect cytosine methylation within this fragment and the fragment F1R4 (SEQ ID NO 1) are listed in tables 2 (together with the according bisulfite converted genomic regions, which are analysed), 5 and 13. Especially preferred are the blocking oligo nucleotides listed in table 2 (SEQ ID NO 4, 46, 48, 50 and SEQ ID NO 52). In examples 3, 4, 5 and 6 the assay is described in more detail.

TABLE 5

List of blockers suitable to inhibit amplification
of unmethylated fragments in assays Exon HM 1 und
Exon HM 2:

| | | |
|---|---|---|
| | | SEQ |
| blocker | | ID |
| notation | DNA sequence | NO |

| | | |
|---|---|---|
| gstp1.10B20 | CCCATCCCCaaaaACaCaaaCCaCa-pho | 4 |
| gstp1.10B22 | CCCATCCCCaaaaACaCaaaCCaC-pho | 80 |
| gstp1.10B23 | CCCATCCCCaaaaACaCaaaCCgC-pho | 81 |
| gstp1.10B24 | CCCATCCCCaaaaACaCGaaCCaC-pho | 82 |
| gstp1.10B25 | CCCATCCCCaaaaACGCaaaCCaC-pho | 83 |
| gstp1.10B26 | CCCATCCCCGaaaACaCaaaCCaC-pho | 84 |
| gstp1.B26.2 | CCCATCCCCCaaaACaCaaaCCaC-pho | 85 |
| gstp1.B26.3 | CCCATCCCCTaaaACaCaaaCCaC-pho | 86 |
| gstp1.10B27 | CCCATCCCCGaaaACGCaaaCCaC-pho | 87 |
| gstp1.10B28 | CCCATCCCCGaaaACaCGaaCCaC-pho | 88 |
| gstp1.10B29 | CCCATCCCCGaaaACaCaaaCCGC-pho | 89 |
| gstp1.10B30 | CCCATCCCCaaaaACGCGaaCCaC-pho | 90 |
| gstp1.10B31 | CCCATCCCCaaaaACGCaaaCCGC-pho | 91 |
| gstp1.10B32 | CCCATCCCCaaaaACaCGaaCCGC-pho | 92 |
| gstp1.10B100 | CATCCCCaaaaACaCaaaCCaCaCaTAC-pho | 93 |
| gstp1.10B101 | ATCCCCaaaaACaCaaaCCaCaCaTAC-pho | 94 |
| gstp1.10B102 | CCATCCCCaaaaACaCaaaCCaCaCaTAC-pho | 95 |
| gstp1.10B103 | CATCCCCaaaaACaCaaaCCaCaCaTA-pho | 96 |
| gstp1.10B105 | CCATCCCCaaaaACaCaaaCCaCaCaTA-pho | 97 |
| gstp1.10B106 | CCCATCCCCaaaaACaCaaaCCaCaCaTA-pho | 98 |
| gstp1.10B107 | CCCATCCCCaaaaACaCaaaCCaCaCaT-pho | 99 |
| gstp1.10B107-G | CCCATCCCCaaaaAaCaaaCCaCaCaT-pho | 100 |
| gstp1.10B117.2 | CCCATCCCCTaaaACACTaaCCACACATACTCA-pho | 101 |
| gstp1.10B118.2 | CCCATCCCCTaaaACACAaaCCTCACATACTCA-pho | 102 |
| gstp1.10B119 | aAaCCCCATCCCCTaaaACACTaaCCACACAT-pho | 103 |
| gstp1.10B120 | aAaCCCCATCCCCTaaaACACAaaCCTCACAT-pho | 104 |

The sequences of the blockers are designated in 5′ to 3′ direction; where pho indicates phosphorylated and ‘a’ instead of A indicates an adenosine residue originated from an unmethylated cytosine in the reverse complementary DNA strand.

TABLE 6

List of detection probes that are suitable to detect
the amplified fragments according to the assays Exon
HM 1 and Exon HM 2 preferably in a Real-Time PCR.

| | | |
|---|---|---|
| | | SEQ |
| probe | | ID |
| notation | DNA sequence | NO |

| | | |
|---|---|---|
| gstp1.10-fluo1 | TTCGtCGtCGtAGTtTTCGtt-fluo | 5 |
| gstp1.10-red1 | red640-tAGTGAGTACGCGCGGtt-pho | 6 |
| gstp1.10-fluo2 | tGAGGttTTtGtTGGAGTTTtGtt-fluo | 105 |
| gstp1.10-red2 | red705- | 106 |
| | tGtAGTtTTtGttAttAGTGAGTAtGtGtG-pho | |
| gstp1.10-fluo5 | GtTGGAGTTTCGtCGtCGt-fluo | 107 |
| gstp1.10-fluo10 | TTCGtCGtCAtAGTtTTCGtt-fluo | 108 |
| gstp1.10-fluo11 | TTCGtCAtCAtAGTtTTCGtt-fluo | 109 |
| gstp1.10-fluo12 | AGTTTCGtCGtCAtAGTtTTCGtt-fluo | 110 |
| gstp1.10-fluo20 | AGTTTCGtCGtCGtAGTtTTCGtt-fluo | 111 |
| gstp1.10- | TTCGtTAtCGtAGTtTTCGtt-fluo | 112 |
| fluo1SNP | | |
| gstp1.10- | TGGAGTTTCGtTAtCGtAGTtTTCGtt-fluo | 113 |
| fluoSNP2 | | |
| gstp1.10-red5 | red640-GTtTTCGttAttAGTGAGTACGCG-pho | 114 |
| gstp1.10-red6 | red640-tAGTGAGTACGCGYGGtt-pho | 115 |
| gstp1.10-red7 | red640-tAGTGAGTACGtGCGGtt-pho | 116 |
| gstp1.10-red20 | red640-tAGTGAGTACGCGCGGttCG-pho | 117 |
| HM 4 Probe fluo | CGTCGTCGTAGTTTTCGTT-fluo | 118 |
| gstp1.12-fluo | CTTCGCCACCAATAAATACGC-fluo | 119 |
| gstp1.12-red | red640-CGaCCCGCGTCCC-pho | 120 |

The sequences of the probes are designated in 5′ to 3′ direction; where fluo indicates fluorescein, pho indicates phosphorylated, red640 indicates LightCycler 640 fluorophor, red705 indicates LightCycler 705 fluorophor, t indicates a thymine residue originated from an unmethylated cytosine and Y indicates C or T.
Exon HM 3:

In one of the preferred assays, assay Exon HM 3 a fragment of 79 bp is generated by the primer pair F1 (SEQ ID NO 2) and R6 (SEQ ID NO 38), which can be located at nt 1845-1924 of GenBank entry AY324387.

Forward gstp1.14 F1:

SEQ ID NO 2

GGGATTATTTTTATAAGGTT
Reverse gstp1.14 R6:

SEQ ID NO 36

TACTCACTAATAACKAAAACTAC
Blocker gstp1.14 B1:

SEQ ID NO 48

CTAATAACAAAAACTACaACaACaAAACTCCAAC

As this fragment is especially short, the use of scorpion primers is preferred, especially when focussing on the CpG positions located in the exon region. The use of such a scorpion primer is described in detail in patent application DE 103 38 308.5. The performance of the assay Exon HM 3 is described in example 8.
Exon EM 4:

This assay is another preferred embodiment characterized by the preferred primer pair and blocker combination as given in tables 1 and 2:

Forward F1:

SEQ ID NO:2

GGGATTATTTTTATAAGGTT
Reverse R8:

SEQ ID NO:40

CTCTAAACCCCATCCCC

Blocker gstp1.10B22: CCCATCCCCaAAAACaCaAACCaC SEQ ID NO 50 (oder 14 oder 80)

The preferred probes are Light Cycler probes:

| | |
|---|---|
| HM4 Probe fluo: | |
| CGTCGTCGTAGTTTTCGTT-fluo | SEQ ID NO: 118 |
| HM4 Probe red: | |
| red640-TAGTGAGTACGCGCGGTT-pho | SEQ ID NO: 6 |

The performance of assay Exon EM 4 is described in more detail in example 7.
Exon HM 5:

In another one of the preferred assays, assay Exon HM 5 the bisulfite a fragment of 91 bp is generated by the primer pair F1 (SEQ ID NO 2) and R2 (SEQ ID NO 43), which can be located at nt 1876-1966 of GenBank entry AY324387. This assay is designed to detect the methylation states of the cytosine bases in the antisense strand of the GSTP1 exon 1. When bisulfiite treated sense and antisense strand differ in their sequence to such an extent that they can not longer be called corresponding. Therefore a different assay design is required. In any case methylated positions will appear as cytosines wherein unmethylated positions will appear as thymine.

The assay is characterized by the preferred primer pair and blocker combination as given in tables 1 and 2:

| | |
|---|---|
| gstp1.12F1: | |
| 5-GTTGGGAGTTTTGAGTTTTATTTT-3 | SEQ ID NO: 42 |
| gstp1.12R2: | |
| 5-AAACCTTCKCTAAAATTTC-3 | SEQ ID NO: 43 |
| gstp1.12B2: | |
| 5-CTAAAATTTCaCCaCCaCAATCTTCaCCAC-3 | SEQ ID NO: 52 |

The preferred LightCycler probes are:

| | |
|---|---|
| gstp1.12-fluo: | |
| CTTCGCCACCAATAAATACGC | SEQ ID NO: 119 |
| gstp1.12-red: | |
| LCred640-CGaCCCGCGTCCC-pho | SEQ ID NO: 120 |

Especially preferred is the combined use of these assays with specific detection probes, especially preferred with real-time detection probes. However, a person skilled in the art will understand that these probes might be replaceable.

One preferred embodiment of this invention (the specific assay Exon HM 1) allows to increase the sensitivity of the Exon HM assay up to a ratio of 1 in 8000 and to enable detection of a single molecule, indicating the methylation of cytosine of one or more relevant CpG sites, within a background of 4.000 or even up to 8.000 molecules of unmethylated background DNA. It is also applicable for detection of a single molecule indicating non-methylated cytosines at one or more relevant CpG sites, within a background of 4.000 or even up to 8.000 molecules of methylated background DNA.

It is preferred that the bisulfite treated (or converted) sequences SEQ ID NOs 2, 35, 37, 39, 41, 42, 44, 45, 47, 49, 51 and SEQ ID NO 53 of the genomic DNA that hybridize to the primer and blocking oligo nucleotides according to SEQ ID NOs 2, 3, 36,38,40,42,43, 4, 46, 48, 50 and 52 (according to tables 1 to 4) are used for the diagnostic analysis of aberrant GSTP1 methylation. It is especially preferred that the bisulfite treated (or converted) sequences SEQ ID NOs 2, 35 and 37 are used for the analysis of aberrant methylation of the GSTP1 gene. The most preferred blocking oligo nucleotides are the nucleic sequences according to SEQ D 4, 46, 48, 50 and 52.

As the blocking oligo nucleotide is directed towards blocking the unmethylated molecules, the CpG sites appear as TpG sites in the sequence complementary to those after treatment with bisulfite as presented SEQ ID 45, 47, 49, 51, and 53.

The invention is also characterized by employing oligo nucleotides that are longer than it has been described before. Blocking oligos with a length of up to 45 nucleotides were shown to have worked successfully in our hands. Therefore, the invention is also characterized in employing oligo nucleotides of a sequence length between 12 and 45 nucleotides.

It is one embodiment of the invention to employ oligo nucleotides of a sequence length between 12 and 45 nucleotides.

It is another preferred embodiment of the invention that said blocking oligo nucleotides are RNA oligonucleotides. The advantage being that said RNA oligonucleotides do not need to be treated to inhibit their extension, because DNA polymerases do not extend RNA molecules.

Also disclosed herein are the means—as shown for the analysis of the example gene GSTP1—on how to improve the HeavyMethyl assay with respect to its reliability and robustness. The invention is characterized by the additional simultaneous amplification of a control fragment in a duplex-PCR experiment within the same real-time PCR reaction tube.

In a preferred embodiment the control fragment, is characterized by being selected out of the genomic region within a certain range of neighboring bps. It is a preferred embodiment of the invention that the control fragment's sequence is not further away from the CpG site analyzed than 2 kb. It is especially preferred, however, that the control fragment's sequence is not further away from the CpG site analyzed than 1 kb.

The method, which is subject of this invention, characterized as employing the improved HeavyMethyl assay for detection of cytosine methylation in DNA samples, and especially within the gene GSTP1, the sequences of the two strands after bisulfite conversion are given in SEQ ID NOs 34 and 35—comprises the following steps:

A genomic DNA sample, which comprises the DNA to be investigated and background DNA is chemically treated in such a way that all of the unmethylated cytosine bases are converted to uracil, whereas the 5-methylcytosine bases remain unchanged;

the chemically treated DNA sample is amplified with the use of at least 2 primer oligonucleotides as well as a polymerase, whereby the DNA to be investigated is preferred as the template over the background DNA, and

the amplified products are analyzed and conclusions are drawn on the methylation status of the DNA to be investigated, from the presence of an amplified product and/or from the analysis of other positions.

It is preferred according to the invention that the sample DNA is obtained from serum or urine or other body fluids of an individual.

It is preferred according to the invention that the sample DNA comprises genomic DNA coding for the GSTP1 protein according to nt 1183 to nt 1309 of Genbank Accession number M24485.1.

It is further preferred according to the invention that sample DNA is obtained from cell lines, blood, sputum, stool, urine, serum, cerebro-spinal fluid, tissue embedded in paraffin, for example, tissue from eyes, intestine, kidneys, brain, heart, prostate, lungs, breast or liver, histological slides, and all possible combinations thereof.

It is most particularly preferred according to the invention that the chemical treatment is conducted with a bisulfite (═disulfite, hydrogen sulfite). It is also preferred that the chemical treatment is conducted after embedding the DNA in agarose. It is also and additionally preferred that a reagent that denatures the DNA duplex and/or a radical-scavenger is present in the chemical treatment.

It is preferred that the amplification is conducted in the second step in the presence of at least one additional oligonucleotide or PNA oligomer, which binds to a 5′-CG-3′ dinucleotide or a 5′-TG-3′ dinucleotide or a 5′-CA-3′ dinucleotide, whereby the additional oligonucleotide or PNA oligomer preferably binds to the background DNA and adversely affects its amplification. This oligonucleotide or PNA oligomer is also referred to as ‘blocking oligo’.

It is particularly preferred that the blocking oligo is a RNA oligonucleotide.

It is also particularly preferred that the binding site of the additional oligonucleotide or PNA oligomer, hence the blocking oligo, overlaps with the binding sites of the primers on the background DNA and the additional oligonucleotide hinders the binding of at least one primer oligonucleotide to the background DNA.

In addition, it is particularly preferred that at least two additional oligonucleotides or PNA oligomers are utilized, whereby their binding sites each overlap in turn with the binding site of one primer on the background DNA, and the additional oligonucleotides and/or PNA oligomers hinder the binding of both primer oligonucleotides to the background DNA.

It is also particularly preferred that one of the additional oligonucleotides and/or PNA oligomers prevents the binding of the forward primer, while the other prevents the binding of the reverse primer.

It is particularly preferred that the additional oligonucleotides and/or PNA oligomers are present in at least five times the concentration of the primer oligonucleotides.

In another particularly preferred variant of the method, the additional oligonucleotides and/or PNA oligomers bind to the background DNA and thus prevent the complete elongation of the primer oligonucleotide in the polymerase reaction. It is particularly the case that the polymerase used does not have 5′-3′ exonclease activity. Another preferred variant is that the additional oligonucleotides present are modified at the 5′ end and thus cannot be significantly broken down by a polymerase with 5′-3′ exonuclease.

In addition, it is preferred according to the invention that the chemically treated DNA sample is amplified in the second step with the use of at least 2 primer oligonucleotides and another oligonucleotide, which hybridizes to a 5′-CG-3′ dinucleotide or a 5′-TG-3′ dinucleotide or a 5′-CA-3′ dinucleotide, and at least one reporter oligonucleotide, which hybridizes to a 5′-CG-3′ dinucleotide or a 5′-TG-3′ dinucleotide or a 5′-CA-3′ dinucleotide, as well as a polymerase; whereby the additional oligonucleotide preferably binds to the background DNA and adversely affects its amplification, and whereby the reporter oligonucleotide binds preferably to the DNA to be investigated and indicates its amplification. It is thus advantageous that another oligomer labeled with a fluorescent dye is used in addition to the reporter oligonucleode so that this other oligomer hybridizes directly adjacent to the reporter oligonucleotide and this hybridization can be detected by means of fluorescence resonance energy transfer. It is further advantageous that a TaqMan assay is conducted. It is also preferable that a LightCycler assay is conducted.

It is further preferred according to the invention that the oligonucleotides used in addition to the primers do not make available a 3′—OH function. In addition, it is preferred that the reporter oligonucleotide bears at least one fluorescent label. It is also preferred that the reporter molecules indicate the amplification either by an increase or a decrease in fluorescence. It is particularly advantageous that the increase or decrease of fluorescence is also directly used for analysis and that the methylation state of the DNA to be analyzed can be concluded from the fluorescent signal.

It is further preferred according to the invention that the background DNA is present in 100 times the concentration of the DNA to be investigated. It is also preferred that the background DNA is present in 1000 times the concentration of the DNA to be investigated.

It is a particularly preferred embodiment of the invention that the background DNA is present in 4000 times the. concentration of the DNA to be investigated. It is also preferred that the background DNA is present in 8000 times the concentration of the DNA to be investigated

It is one particularly preferred embodiment of this invention that the CpG sites investigated are located within the nucleic acid defined by the sequence nt 1183 to nt 1309 of Genbank Accession number M24485 1.

It is also advantageous according to the invention that the oligomers hybridize to the DNA to be analyzed by means of a 12-45 base long segment and that these include a CG, TG or CA dinucleotide.

It is one especially advantageous feature of the assay described in this invention that it offers the flexibility to analyze a variable number of CpG sites. In this it is a highly flexible method that can be used to differentiate methylation patterns on the basis of only three differentially methylated CpG sites or on the basis of up to 40 or even in rare cases up to 60 differentially methylated CpG sites in one experiment.

It is therefore preferred that the methylation status of several CpG positions than 20 methylation positions of the GSTP1 gene and its regulatory region is is detected in one experiment.

It is additionally preferred that the methylation status of more that 60 methylation positions of the DNA to be analyzed is detected in one experiment.

It is also preferred that the presence of a cell proliferative disorder of the patient or individual the DNA sample is obtained from is concluded from the methylation degree of the different CpG positions located within the GSTp1 gene or its regulatory region which are investigated with the method according to the invention.

It is further particularly preferred that the method according to the invention is used to early detect a cell proliferative disorder in a screening procedure or to monitor cell proliferative disorders which are associated with aberrant methylation of the GSTP1 gene or its regulatory region.

It is furthermore particularly preferred that the method according to the invention is used to early detect a prostate cancer or to distinguish prostate cancer from BPH (benign prostate hyperplasia) in a screening procedure or to monitor cell proliferative disorders which are associated with aberrant methylation of the GSTP1 gene or its regulatory region.

A particularly preferred embodiment of the invention is finally a kit which is characterized as comprising at least one primer oligo nucleotide out of the group consisting of SEQ ID NOs: 2, 42, 54, 58, 62 and 66 and one primer (reverse) out of the group consisting of SEQ ID NOs: 3, 36, 38, 40, 43, 56, 60, 64 and 68 and at least one blocker out of the group of SEQ ID NO 4, 46, 48, 50, 52, 70, 72, 74, 76, 80, 81, 82, 83, 84, 85, 86, 87, 88, 89, 90, 91, 92, 93, 94, 95, 96, 97, 98, 99, 100, 102, 103 and SEQ ID NO:104.

It is preferred that the kit comprises at least one of the blocking oligo nucleotides out of the group consisting of SEQ ID NO 4, 46, 48, 50, 52, 70, 72, 74, and SEQ ID NO 76.

It is particularly preferred that each kit comprises all three necessary components according to the invention related to at least one assay, according to Tables 1-4.

A kit comprising the oligo nucleotides of the group consisting of SEQ ID NOS 2, 3 and 4 is therefore a particularly preferred embodiment.

A kit comprising the oligo nucleotides of the group consisting of SEQ ID NOS 2, 36 and 46 is therefore a particularly preferred embodiment.

A kit comprising the oligo nucleotides of the group consisting of SEQ ID NOS 2, 38 and 48 is therefore a particularly preferred embodiment.

A kit comprising the oligo nucleotides of the group consisting of SEQ ID NOS 2, 40 and 50 is therefore a particularly preferred embodiment.

A kit comprising the oligo nucleotides of the group consisting of SEQ ID NOS 2, 40 and 50 is therefore a particularly preferred embodiment.

### EXAMPLES

### Example 1

Detection of methylated DNA using the HeavyMethyl GSTP1 (Exon 1) assay Exon HM 1.

Genomic DNA was isolated from the samples and treated with a solution of bisulfite as it is described in Olek et al. Nucleic Acids Res. 1996 Dec. 15;24(24):5064-6. As a result of this treatment cytosine bases that were unmethylated were converted to thymine and are in the following indicated as such by the use of small t instead of capital T which respectively stands for a thymine base, that was a thymine base prior to treatment with bisulfite.

The HeavyMethyl assay of the GSTP1 (Exon 1) fragment F1R4 (nt 1183 to nt 1309 in Genbank Accession M24485.1) was performed in a total volume of 20 μl using a LightCycler device (Roche Diagnostics). The real time PCR reaction mix contained 10 μl of template DNA (for concentrations see below), 2 μl of FastStart LightCycler reaction mix for hybridization probes (Roche Diagnostics, Penzberg), 0.30 μM forward primer (SEQ ID NO: 2; GGGAttAtttTTATAAGGtT), 0.30 μM reverse primer (SEQ ID NO: 3; CCATACTaaaAaCTCTaAaCCC), 0.15 μM fluorescein anchor probe (SEQ ID NO: 5; TTCGtCGtCGtAGTtTTCGtt-fluorescein; TIB-MolBiol, Berlin), 0.15 μM detection probe (SEQ ID NO: 6; red640-tAGTGAGTACGCGCGGtt-phosphate; TIB-MolBiol, Berlin), 1 μM blocker oligonucleotide (SEQ ID NO: 4; CCCATCCCCaAAAACaCaAACCaCa) and 3mM MgCl₂ and. Thermocycling conditions began with a 95 degree C. incubation for 10 minutes, then 55 cycles of the following steps: 95 degrees C. for 10 seconds, 56 degrees C. for 30 seconds, and 72 degrees C. for 10 seconds. Fluorescence was detected after the annealing phase at 56 degrees C. in each cycle.

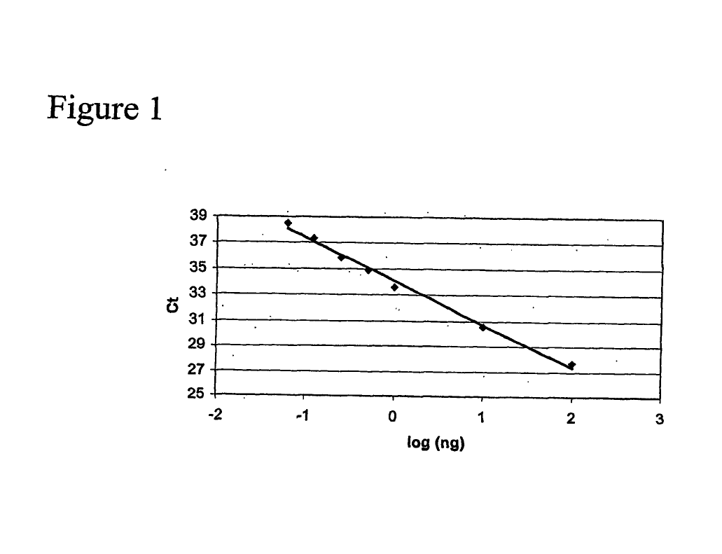

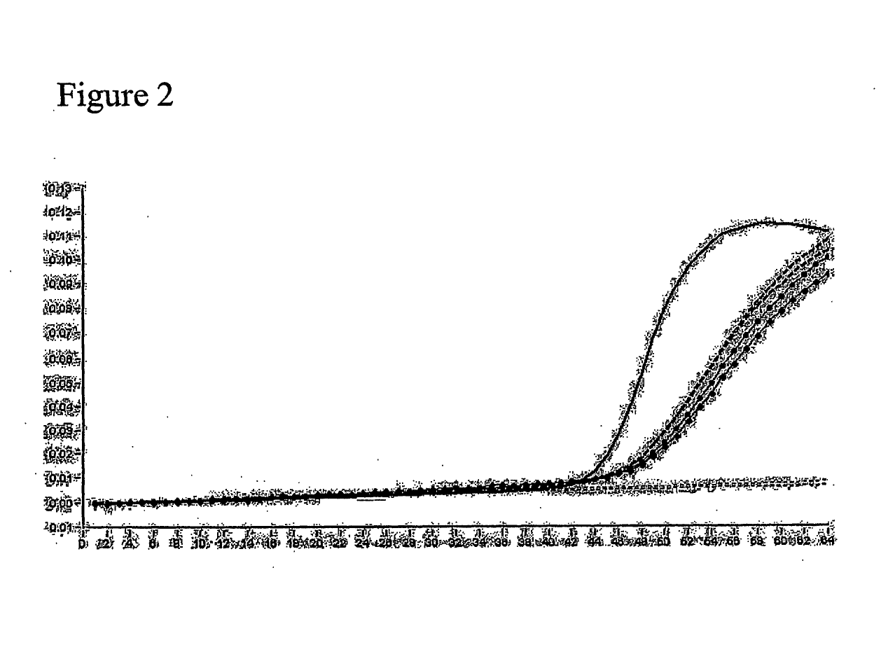

As template DNA bisulfite treated human DNA isolated from peripheral blood cells and commercially available bisulfite treated human DNA, which was methylated enzymatically (provided by Serologicals), was used. The amount of DNA after bisulfite treatment was measured by UV absorption at 260 nm. The performance of the assay on 100 ng, 10 ng, 1 ng, 0.5 ng, 0.25 ng, 0.125 ng and 0.0625 ng bisulfite treated methylated template DNA was analyzed. Table 1 shows the mean of the cycle threshold values of 4 replicates as calculated by the LightCycler software. The data indicate, that the assays show a linearity of at least 4 orders of magnitude on bisulfite treated methylated template DNA (FIG. 1). The absolute analytical sensitivity of the assay was found to be at least 25 pg bisulfite treated methylated template DNA. The relative analytical sensitivity was deternined using 100 pg and 50 pg bisulfite treated methylated template DNA spiked into 400 ng bisulfite treated non-methylated template DNA. Amplificates (SEQ ID NO: 1) at the relative sensitivity values of 1:4000 and 1:8000 were obtained, whereas no amplificates on 400 ng bisulfite treated non-methylated DNA was generated (FIG. 2).

TABLE 7
Performance of HeavyMethyl GSTP1 (Exon 1) assay

| amount of bisulfite | Mean of 4 threshold |
|---|---|
| treated methylated | cycles obtained from |
| template DNA (ng) | 4 replicated experiments |
| 100 | 27.7 |
| 10 | 30.51 |
| 1 | 33.58 |
| 0.5 | 34.94 |
| 0.25 | 35.92 |
| 0.125 | 37.38 |
| 0.0625 | 37.88 |

### Example 2

Duplex PCR of a methylation unspecifc control fragment and GSTP1 (exon 1) using the HeavyMethyl assay Exon HM 1.

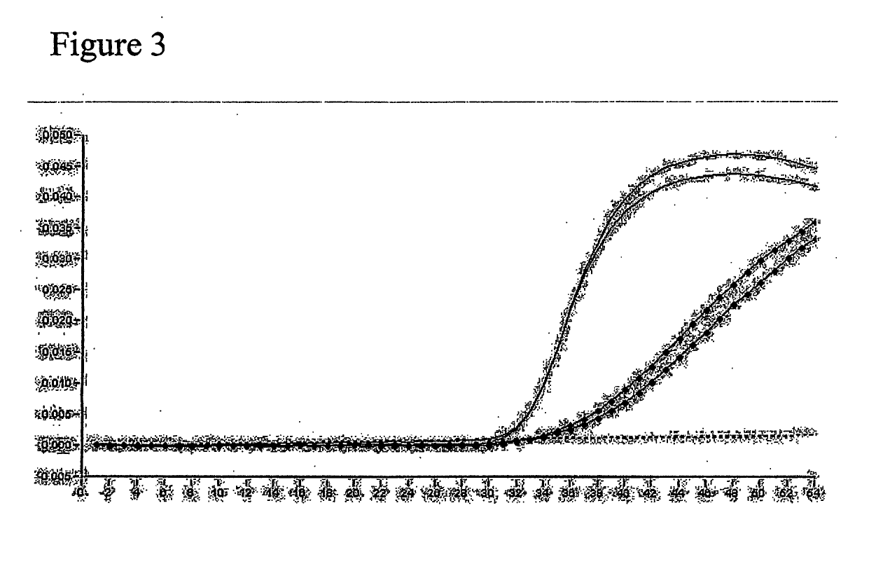

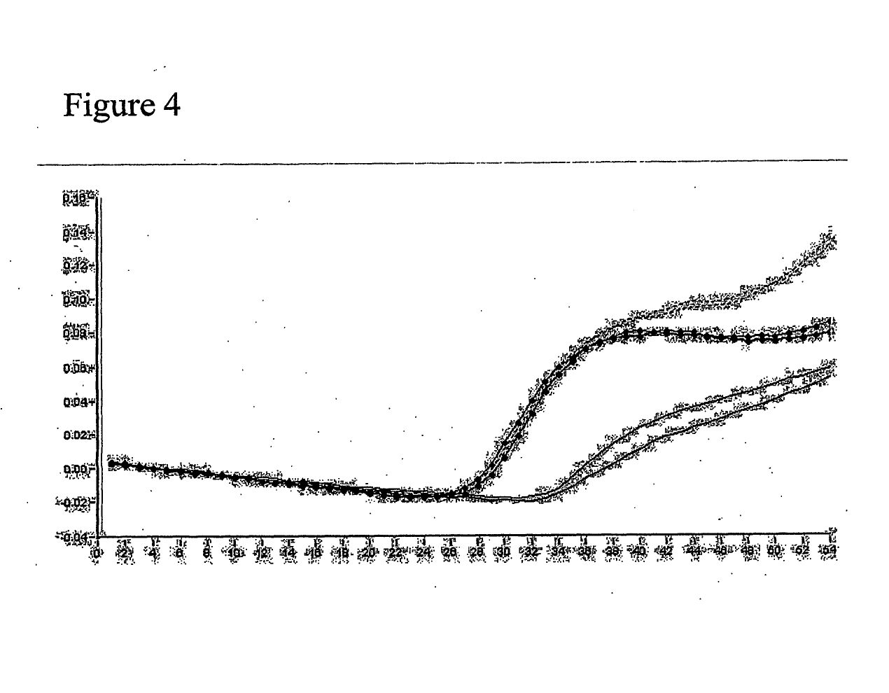

In this example the HeavyMethyl assay of the GSTP1 Exon 1 was combined with a methylation unspecific PCR of a control fragment. The control fragment is located in the GSTP1 intron 4 region and comprises nt 2273 to nt 2303 in Genbank Accession M24485.1. This real time duplex PCR was performed in a total volume of 20 μl using a LightCycler device (Roche Diagnostics). The real time PCR reaction mix contained 2 μl of FastStart LightCycler reaction mix for hybridization probes (Roche Diagnostics, Penzberg), 0.60 μM forward primer (SEQ ID No: 2; GGGAttAtttTTATAAGGtT), 0.60 μM reverse primer (SEQ ID NO: 3; CCATACTaaaAaCTCTaAaCCC), 0.15 μM anchor probe (SEQ ID NO: 5; TTCGtCGtCGtAGTtTTCGtt-fluorescein; TIB-MolBiol, Berlin), 0.15 μM detection probe (SEQ ID 6; red640-tAGTGAGTACGCGCGGtt-phosphate; TIB-MolBiol, Berlin), 2 μM blocker oligonucleotide (SEQ ID No: 4; CCCATCCCCaAAAACaCaAACCaCa), 0.075 μM control forward primer (SEQ ID No: 8; GGAGTGGAGGAAAtTGAGAt), 0.075 μM control reverse primer (SEQ ID No: 9; CCACACAaCAaaTaCTCAaAaC), 0.15 μM control fragment anchor probe (SEQ ID No: 10; GtttAAGGTtAAGttTGGGTGttTGtA-fluorescein; TIB-MolBiol, Berlin), 0.15 μM control fragment detection probe (SEQ ID NO: 11; ttTTGtttTGTGttAGGtTGttTtttAGG; TIB-MolBiol, Berlin), and 3mM MgCl₂. Thermocycling conditions began with a 95 degree C. incubation for 10 minutes, then 55 cycles of the following steps: 95 degrees C. for 10 seconds, 56 degrees C. for 30 seconds, and 72 degrees C. for 10 seconds. Fluorescence was detected after the annealing phase at 56 degrees C. in each cycle. The amplification of the resulting GSTP1 (exon 1) fragment F1R4 (SEQ ID NO: 1) and the control fragment (SEQ ID NO: 7) were monitored using the F2/F1 (FIG. 3) and F3/F2 (FIG. 4) analyzing mode of the LightCycler software, respectively. The performance of this assay was analyzed on bisulfite treated methylated DNA, bisulfite treated non-methylated DNA, and mixtures thereof. Said GSTP1 (exon 1) fragment was detected using 100 pg methylated or 500 pg methylated bisulfite treated DNA spiked into 100 ng non-methylated bisulfite treated template DNA. No amplificate was found, when 100 ng non-methylated bisulfite treated DNA was used as template DNA. The control fragment simultaneously amplified with said GSTP1 (exon 1) fragment and monitored in the F3/F2 channel was amplified independent of the methylation status using 1000 pg methylated, 100 ng non-methylated or a mixture of 500 pg methylated and 100 ng non-methylated bisulfite treated DNA.

The data show that the established duplex PCR enables the quantitative determination of the amount of GSTP1 sequence methylated prior to bisulfite treatment, by methylation specific amplification of the GSTP1 fragment (SEQ ID NO: 1). The additional determination of the total amount of template DNA was achieved by employing said GSTP1 control fragment as template in a simultaneously performed control PCR in the same real time PCR tube.

### Example 3

Comparison of Light Cycler and Taqman Detection of methylated DNA using the HeavyMethyl GSTP1 (Exon 1) assay exon HM 2.

Genomic DNA was isolated from the samples and treated with a solution of bisulfite as it is described in Olek et al. Nucleic Acids Res. 1996 Dec. 15;24(24):5064-6. As a result of this treatment cytosine bases that were unmethylated were converted to thymine and are in the following indicated as such by the use of small t instead of capital T which respectively represents a thymine base, that was a thymine base prior to treatment with bisulfite.

Light Cycler Assay:

The HeavyMethyl assay of the GSTP1 (Exon 1) fragment (nt 1183 to nt 1303 in Genbank Accession M24485.1) was performed in a total volume of 20 μl using a LightCycler device (Roche Diagnostics). The real time PCR reaction mix contained 10 μl of template DNA (for concentrations see below), 2 μl of FastStart LightCycler reaction mix for hybridization probes (Roche Diagnostics, Penzberg), 0.30 μM forward primer (SEQ ID NO: 2; GGGAttAtttTTATAAGGtT), 0.30 μM reverse primer (SEQ ID NO: 12; TaCTaaaAaCTCTaAaCCCCATC), 0.15 μM fluorescein anchor probe (SEQ ID NO: 5; TTCGtCGtCGtAGTtTTCGtt-fluorescein; TIB-MolBiol, Berlin), 0.15 μM detection probe (SEQ ID NO: 6; red640-tAGTGAGTACGCGCGGtt-phosphate; TIB-MolBiol, Berlin), 4 μM of one of the blocker oligonucleotides listed in table 2 (gstp1.B18, gstp1.B19,gstp1.B20, gstp1.B21, gstp1.B22) and 3.5 mM MgCl₂. Thermocycling conditions began with a 95 degree C. incubation for 10 minutes, then 55 cycles of the following steps: 95 degrees C. for 10 seconds, 56 degrees C. for 30 seconds, and 72 degrees C. for 10 seconds. Fluorescence was detected after the annealing phase at 56 degrees C. in each cycle.

Taqman Assay:

The HeavyMethyl assay of the GSTP1 (Exon 1) fragment (nt 1183 to nt 1303 in Genbank Accession M24485.1) was performed in a total volume of 20 μl using a Taqman 7700 device (ABI). The real time PCR reaction mix contained 10 μl of template DNA (for concentrations see below), 2 μl of FastStart LightCycler reaction mix for hybridization probes (Roche Diagnostics, Penzberg), 0.30 μM forward primer (SEQ ID NO: 2; GGGAttAtttTTATAAGGtT), 0.30 μM reverse primer (SEQ ID NO: 13; TaCTaaaAaCTCTaAaCCCCATC), 0.3 μM of one of Taqman probes (Taq1, Taq2, Taq3, Taq4) listed in table 3, 4 μM of one of the blocker oligonucleotides (gstp1.B18, gstp1.B19, gstp1.B20, gstp1.B21, gstp1.B22) listed in table 2 and 3.5 mM MgCl₂. Thermocycling conditions began with a 95 degree C. incubation for 10 minutes, then 55 cycles of the following steps: 95 degrees C. for 10 seconds, 56 degrees C. for 30 seconds, and 72 degrees C. for 10 seconds. Fluorescence was detected after the annealing phase at 56 degrees C. in each cycle.

In both cases, bisulfite treated human DNA isolated from peripheral blood cells and commercially available bisulfite treated human DNA, which was methylated enzymatically (provided by Serologicals), was used as template DNA. The amount of DNA after bisulfite treatment was measured by UV absorption at 260 nm. The performance of the assay on 100 pg bisulfite treated methylated template DNA was analyzed. Table 3 and 4 show the mean of the cycle threshold values of 2 replicates as calculated by the LightCycler software or Taqman 7700 software, respectively. The absolute analytical sensitivity of the assay was found to be at least 100 pg bisulfite treated methylated template DNA. The relative analytical sensitivity was determined using 100 pg bisulfite treated methylated template DNA spiked into 100 ng bisulfite treated non-methylated template DNA. Amplificates (SEQ ID NO: 12) at the relative sensitivity values of 1:1000 were obtained, whereas no amplificates on 100 ng bisulfite treated non-methylated DNA was generated. The mean of the cycle threshold values of 2 replicates as calculated by the LightCycler software or Taqman 7700 software are given in Tables 4 and 5, respectively.

TABLE 8

Sequences of blocker nucleotides

| | |
|---|---|
| Name | Sequence |

| | |
|---|---|
| gstp1.B18 - | 5′-CATCCC CaAAAACaCaAACCaCaCaT |
| SEQ ID 14 | |
| gstp1.B19 - | 5′-CCATCCC CaAAAACaCaAACCaCaC |
| SEQ ID 15 | |
| gstp1.B20 - | 5′-CCCATCCC CaAAAACaCaAACCaCa |
| SEQ ID 16 | |
| gstp1.B21 - | 5′-TCCC CaAAAACaCaAACCaCaCaTA |
| SEQ ID 17 | |
| gstp1.B22 - | 5′-CCCATCCC CaAAAACaCaAACCaC |
| SEQ ID 18 | |
| gstp1.B100 - | 5′-CATCCC CaAAAACaCaAACCaCaCaTAC |
| SEQ ID 19 | |
| gstp1.B101 - | 5′-ATCCC CaAAAACaCaAACCaCaCaTAC |
| SEQ ID 20 | |
| gstp1.B102 - | 5′-CCATCCC CaAAAACaCaAACCaCaCaTAC |
| SEQ ID 21 | |
| gstp1.B103 - | 5′-CATCCC CaAAAACaCaAACCaCaCaTA |
| SEQ ID 22 | |
| gstp1.B104 - | 5′-ATCCC CaAAAACaCaAACCaCaCaTA |
| SEQ ID 23 | |
| gstp1.B105 - | 5′-CCATCCC CaAAAACaCaAACCaCaCaTA |
| SEQ ID 24 | |
| gstp1.B106 - | 5′-CCCATCCC CaAAAACaCaAACCaCaCaTA |
| SEQ ID 25 | |
| gstp1.B107 - | 5′-CCCATCCC CaAAAACaCaAACCaCaCaT |
| SEQ ID 26 | |

Underlined nucleotides indicate overlap to primer, all blocker carry 3′-phosphate modification.

TABLE 9

Sequences of TaqMan detection probes

| | |
|---|---|
| Name | Sequence |

| | |
|---|---|
| Taq1 - | 5′-6FAM- CCg AAA ACg CgA ACC gCg CgT ACT- |
| SEQ ID 27 | DQ |
| Taq2 - | 5′-6FAM- CAC TAA TAA CgA AAA CTA CgA CgA |
| SEQ ID 28 | CgA AAC TT -DQ |
| Taq3 - | 5′-6FAM- AAA Acg CgA ACC gCg CgT ACT C - |
| SEQ ID 29 | DQ |
| Taq4 - | 5′-6FAM- AAC CgC gCg TAC TCA CTA ATA Acg |
| SEQ ID 30 | A -DQ |
| Taq5 - | 5′-6FAM- TCA CTA ATA ACG AAA ACT ACG ACG |
| SEQ ID 31 | ACG AAA CT -DQ |
| Taq6 - | 5′-6FAM- CGC GTA CTC ACT AAT AAC GAA AAC |
| SEQ ID 32 | TAC GAC GAC GA -DQ |
| Taq7 - | 5′-6FAM- TGG AGT TTC GTC GTC GTA GTT TTC |
| SEQ ID 33 | GTT ATT AGT -DQ |

6FAM = fluorescein, DQ = dabcyl

TABLE 10
Performance of HeavyMethyl GSTP1 (Exon 1) LightCycler assay

| | without | Blocker | Blocker | Blocker | Blocker | Blocker |
|---|---|---|---|---|---|---|
| | blocker | B18 | B19 | B20 | B21 | B22 |
| 100 pg | 34 | 35.75 | 35.5 | 35 | 38.2 | 35.85 |
| methylated | | | | | | |
| DNA | | | | | | |
| 1 in 1000 | nd | 34.5 | 35.8 | 37 | 39.2 | 35.9 |
| 100 ng | nd | — | — | — | — | — |
| unmethylated | | | | | | |
| DNA | | | | | | |

TABLE 11
Performance of HeavyMethyl GSTP1 (Exon
1) TaqMan assay using Taq1 probe

| | without | Blocker | Blocker | Blocker | Blocker | Blocker |
|---|---|---|---|---|---|---|
| | blocker | B18 | B19 | B20 | B21 | B22 |
| 100 pg | 36 | 36 | 36 | 36 | 36 | 36 |
| methylated | | | | | | |
| DNA | | | | | | |
| 1 in 1000 | nd | — | 36.8 | 36.5 | — | 37 |
| 100 ng | nd | — | — | — | — | — |
| unmethylated | | | | | | |
| DNA | | | | | | |

### Example 4

Comparison of GSTP1 HeavyMethyl assays Exon HM1 and Exon HM 2 assay on model template DNA.

GSTP1 Exon HM1 assay (nt 1183 to nt 1309 in Genbank Accession M24485.1) was performed in 20 μl, that contained 3 mM MgCl₂ (Roche Diagnostics), 1× LightCycler-FastStart Master Hybridization Probes reaction mix (Roche Diagnostics), 0.3 μM forward primer (GGGATTATTTTTATAAGGTT), 0.3 μM reverse primer (CCATACTAAAAACTCTAAACCC), 1 μM blocker (CCCATCCCCAAAAACACAAACCACA-pho), 0.15 μM donor probe (TTCGTCGTCGTAGTTTTCGTT-fluo), 0.15 μM acceptor probe (LCred640-TAGTGAGTACGCGCGGTT-pho) and 10 ml template DNA.

GSTP1 Exon HM 2 assay (nt 1183 to nt 1306 in Genbank Accession M24485.1) was performed in 20 μl, that contained 3.5 mM MgCl₂ (Roche Diagnostics), 1× LightCycler-FastStart Master Hybridization Probes reaction mix (Roche Diagnostics), 1 μM forward primer (GGGATTATTTTTATAAGGTT), 0.3 μM reverse primer (TACTAAAAACTCTAAACCCCATC), 4 μM blocker (CCCATCCCCAAAAACACAAACCACACAT-pho), 0.15 μM donor probe (TTCGTCGTCGTAGTTTTCGTT-fluo) and 0.15 μM acceptor probe (LCred640-TAGTGAGTACGCGCGGTT-pho) (SEQ ID 6) and 10 ml template DNA.

The cycling conditions for both assays were an initial denaturation step at 95° C. (10 min), then 55 cycles of the following steps: 95° C. denaturation (10 sec), 56° C. annealing (30 sec), 72° C. elongation (10 sec), and a final cooling step of 4° C. The temperature transition rate was 20° C./s in all steps, a single detection step was done at the end of the annealing step during the cycling program. The performance of GSTP1 assays Exon HM 1 and exon HM 2 were compared on the basis of their relative sensitivity on DNA solutions containing 50 pg, 25 pg, 18 pg, 10 pg, 8 pg, 6 pg, 4 pg and 0 pg methylated bisulfite treated DNA spiked in 50 ng unmethylated bisulfite treated DNA, respectively. All PCRs were done in 8 replicates. The number of positive replicates and the mean of their cycle numbers above threshold are indicated in Table 12.

TABLE 12
Relative Sensitivity of Exon HM 1 and Exon HM 2 Assay.

| amount of | | | | | |
|---|---|---|---|---|---|
| spiked | | Exon HM 1 assay | | Exon HM 2 assay | |
| methylated | total | no of | | no of | |
| DNA per PCR | no of | replicates | mean | replicates | mean |
| reaction [pg] | samples | detected | CP | detected | CP |
| 50 | 8 | 8 | 38.6 | 8 | 36.5 |
| 25 | 8 | 8 | 40.2 | 8 | 37.2 |
| 18 | 8 | 7 | 41.6 | 7 | 36.8 |
| 10 | 8 | 3 | 41.3 | 7 | 37.9 |
| 8 | 8 | 5 | 40.6 | 5 | 37.2 |
| 6 | 8 | 5 | 40.9 | 5 | 38.1 |
| 4 | 8 | 1 | 40.6 | 4 | 37.6 |
| 0 | 8 | 0 | — | 0 | — |

### Example 5

Analysis of the methylation state GSTP1 Exon 1 to test the performance of assay GSTP1 Exon HM 2 on DNA isolated from tissue samples

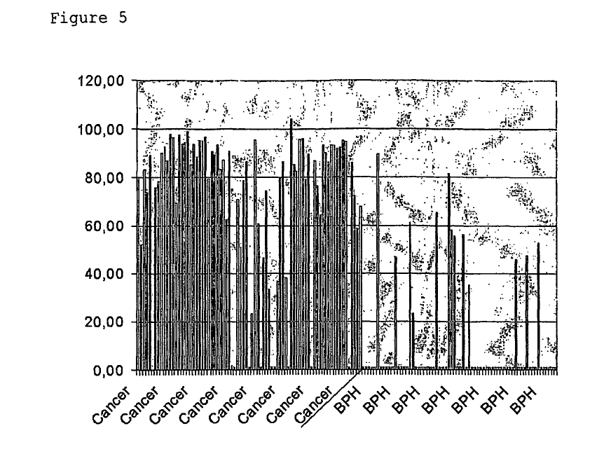

DNA from the biopsie samples was prepared using Qiagen Amp MiniKit (Qiagen, Hilden) and subsequently treated with bisufite as described by Olek et al (see above). Appr. 10 ng DNA was analysed with GSTp1 exon HM 2 assay and the amount of methylated DNA was calculated by comparision of a standard curve of different amounts of completely methylated bisulfite treated DNA. The relative methylation values were determined as ratio of methylated GSTp1 exon 1 DNA and total amount of bisulfite DNA, determined by a methylation unspecific but bisulfite DNA specific real time PCR. The relative methylation values of tumor and BPH samples are shown in FIG. 5. The sensitivity and specificity of GSTp1 HM 2 assay was determined as 86.1% and 80%, respectively. This is a very good performance and might well serve as the basis for a prescreening test in body fluids.

### Example 6

Blocker optimization of assay Exon HM 2.

For the optimization of the blocker performance in assay Exon HM 2 the performance of the following blocker oligonucleotides was analyzed. The blockers vary in sequence, total length, Tm and overlap with the reverse primer:

TABLE 13

| blocker | | Tm | overlap with | |
|---|---|---|---|---|
| name | sequence¹⁾ | [° C.]²⁾ | reverse primer | length |
| B20 | CCCATCCCCaaaaACaCaaaCCaCa-pho | 62.2 | 6 bp | 25 bp |
| B100 | CATCCCCaaaaACaCaaaCCaCaCaTAC-pho | 60.8 | 4 bp | 28 bp |
| B101 | ATCCCCaaaaACaCaaaCCaCaCaTAC-pho | 60.2 | 3 bp | 27 bp |
| B102 | CCATCCCCaaaaACaCaaaCCaCaCaTAC-pho | 62.4 | 5 bp | 29 bp |
| B103 | CATCCCCaaaaACaCaaaCCaCaCaTA-pho | 60.2 | 4 bp | 27 bp |
| B105 | CCATCCCCaaaaACaCaaaCCaCaCaTA-pho | 61.9 | 5 bp | 28 bp |
| B106 | CCCATCCCCaaaaACaCaaaCCaCaCaTA-pho | 63.5 | 6 bp | 29 bp |
| B107 | CCCATCCCCaaaaACaCaaaCCaCaCaT-pho | 64.0 | 6 bp | 28 bp |

¹⁾underlined oligonucleotides indicate sequence shared with the primer, pho indicates phosphorylated.
²⁾Melting temperature (Tm) calculated with Oligo Analyzer 3.0 (IDT BioTools)

PCR was performed according to the assay exon HM 2 assay with optimized PCR conditions as described in example 4 the primers gstp1.10F1 and gstp1.10R5 with no and each of the 8 different blockers. Methylated DNA was detected with the probes gstp1.10-fluo1 and gstp1.10-red1 while unmethylated DNA was detected with the probe pair gstp1.10-fluo2, gstp1.10-red2. Specificity of both probe pairs was verified on methylated and unmethylated DNA.

Potential inhibition of the blockers on the amplification of methylated DNA was tested on 100 pg methylated DNA (see table 14). The mean of the CPs of the PCR on 100 pg methylated DNA without and with different blockers was 35.8 and 36.1 to 36.6, respectively. Since the maximal standard deviation for the CP values of the replicates was 0.66, it can be concluded that no major differences in the amplification of 100 pg methylated DNA exist in the presence of the 8 different blockers regarding the CP values on methylated DNA template.

TABLE 14
Mean CP Values of Blockers B20 and B100
to B107 on 100 pg methylated DNA

| | mean CP ± SD ¹⁾ for detection |
|---|---|
| blocker | of 100 pg +CH₃ DNA |
| B20 | 36.4 ± 0.66 |
| B100 | 36.1 ± 0.49 |
| B101 | 36.5 ± 0.25 |
| B102 | 36.6 ± 0.61 |
| B103 | 36.4 ± 0.22 |
| B105 | 36.2 ± 0.10 |
| B106 | 36.1 ± 0.16 |
| B107 | 36.1 ± 0.10 |
| none | 35.8 ± 0.45 |

¹⁾ mean CP ± SD indicates mean crossing point obtained from 3 replicates and corresponding standard deviation

The blocking effect on the amplification of unmethylated DNA and the effect on the amplification of methylated DNA in the presence of unmethylated DNA was investigated on 100 pg methylated DNA spiked into 100 ng unmethylated DNA Since the amplification curves on methylated and on methylated DNA spiked into unmethylated DNA have an unequal slope, the crossing points were determined with the fit point method and used as a measure for the incline of the curve. Doing so, high CP values corresponded to a flat amplification curve and vice versa.
Detection of unmethylated DNA yielded amplification curves with a gradual incline in the presence of blockers in comparison to the curve obtained in the absence of a blocker. Correspondingly, higher CP (crossing point) values were observed for the amplification curves in the presence of blockers when compared to the CP values obtained in the absence of a blocker. The blockers can be grouped according to their CP values (slope of amplification curves) in blockers.B100, B101, B103 with equal or worse blocking performance than B20 and into blockers B102, B105, B106, B107 with better blocking performance than B20 (see table 15).
Detection of methylated DNA yielded amplification curves with a much higher signal intensity and steeper progression in the presence of a blocker compared to the PCR in the absence of a blocker. Steepest curve progression and accordingly lowest CP values showed the blockers B20, B105, B106 and B107 (see table 15).

In this study, better performance was observed with blockers with higher Tm and larger overlap to the reverse primer. The best performance showed the blocker B107. This GSTP1 assay named Exon HM 2 specified as follows: 3.5 mM MgCl₂, 1 mM forward primer gstp1.10F1, 0.3 mM reverse primer gstp1.10R5 and 4 μM blocker B107 is an especially preferred embodiment of this invention.

TABLE 15
Mean CP Values of Blockers B20 and B100-B107 on 100
pg methylated DNA Spiked into 100 ng unmethylated DNA

| | mean CP ± SD ¹⁾ for detection | mean CP ¹⁾ for detection |
|---|---|---|
| | of −CH₃ DNA on 100 pg | of +CH₃ DNA on 100 pg |
| | methylated DNA spiked into | methylated DNA spiked into |
| blocker | 100 ng unmethylated DNA | 100 ng unmethylated DNA |
| B20 | 41.8 ± 0.5 | 40.0 ± 0.2 |
| B100 | 40.8 ± 0.4 | 42.8 ± 0.2 |
| B101 | 40.0 ± 1.8 | 42.5 ± 0.5 |
| B102 | 45.5 ± 0.3 | 42.0 ± 0.3 |
| B103 | 40.0 ± 1.6 | 41.2 ± 0.3 |
| B105 | 43.3 ± 2.0 | 40.8 ± 1.0 |
| B106 | 42.9 ± 0.1 | 40.0 ± 1.0 |
| B107 | 42.8 ± 0.5 | 39.8 ± 0.1 |
| none | 31.7 ± 0.4 | 49.3 ± 0.9 |

mean CP ± SD indicates mean crossing point obtained from 3 replicates and corresponding standard deviation

### Example 7

Exon HM 4 performance on body fluid samples

The objective of the following study was to analyze the methylation status of prostate cancer markers in different body fluid samples in order to identify the preferred choice of body fluid (urine or serum) for testing and the preferred marker, markers or combinations of markers. The study was run on matched serum and urine sediment samples from 80 patients with an average age of 65 and representative of a number of racial types (Caucasian, African American etc.). In each case, genomic DNA was analyzed using the HeavyMethyl or MSP technique after bisulfite conversion.

Urine Sediment was prepared for analysis and bisulphite treated according to the following:
200 ul sediment samples were purified using the Magnapure DNA Isolation Kit 1 with a 100 ul elution volume.
5 ul HD6 PCR was carried out on the Magnapure Eluate, in order to determine DNA concentration
100 ul of the DNA solution was treated using a proprietary bisulfite treatment technique
10 ul C3 bisulfite specific quantitative PCR
5 ul Merck sulfite test

Serum was prepared for analysis and bisulphite treated according to the following:
1 mL serum samples were purified using the Magnapure DNA Large Volume Total nucleic acid with a 100 ul elution volume.
5 ul HD6 PCR on Magnapure Eluate—To determine DNA concentration
100 ul of the DNA solution was treated using a proprietary bisulfite treatment technique
10 ul C3 bisulfite specific quantitative PCR
5 ul Merck sulfite test

Single PCR runs were performed on 10 ul of bisulfite treated DNA per sample for each of the markers as described below.

Heavy Methyl Assay of the GSTP1 gene

In the following analysis the methylation status of the gene GSTP1 was analysed by means of a Heavy Methyl assay using the primers according to Table 16 (below).

The sequence of interest is amplified by means of primers and a blocker oligonucleotide in order to minimise the unspecific amplification of non methylated DNA. The amplificate is then detected by means of methylation specific Lightcycler probes.

TABLE 16

Oligonucleotides for Heavy Methyl analysis of
GSTP1.

| | | |
|---|---|---|
| SEQ ID NO: | Sequence | Type |

| | | |
|---|---|---|
| 2 | gggattatttttataaggtt | primer |
| 40 | ctctaaaccccatcccc | primer |
| 50 | cccatccccaaaaacacaaaccac | blocker |
| 118 | CGtCGtCGtAGTtTTCGtt-fluo | probe |
| 6 | red640-tAGTGAGTACGCGCGGtt-pho | probe |

Reaction conditions:

PCR program

| | | | |
|---|---|---|---|
| denat at 95° C. | | | |
| 95° C. | 10 min | | |
| 50 cycles: | | ramp | |
| denat at 95° C. | 10 sec | (1° C./s) | |
| annealing 56 C. ° C. | 30 sec | (1° C./s) | detection |
| extension 72° C. | 10 sec | (1° C./s) | |

Results were analyzed qualitatively by scoring amplification as ± and quantitatively by determining the percentage of methylated DNA as a fraction of total DNA calculated using the C3 bisulfite specific PCR. To measure total methylated DNA, a 100% methylated standard (chemicon SSS1 treated DNA) standard curve was included in each assay.

Results

For each marker a Receiver Operating Characteristic curve (ROC curve) of the assay was determined. A ROC is a plot of the true positive rate against the false positive rate for the different possible cut-points of a diagnostic test. It shows the tradeoff between sensitivity and specificity depending on the selected cut-point (any increase in sensitivity will be accompanied by a decrease in specificity). The area under an ROC curve (AUC) is a measure for the accuracy of a dignostic test (the larger the area the better, optimum is 1, a random test would have a ROC curve lying on the diagonal with an area of 0.5; for reference: J. P. Egan Signal Detection Theory and ROC Analysis, Academic Press, New York, 1975).

AUC results:

| | |
|---|---|
| Serum: ASSAY GSTP1 Exon HM 4 | AUC: 0.51 |
| Urine: ASSAY GSTP1 Exon HM 4 | AUC: 0.58 |

### Example 8

Methylation analysis of the GSTp1 gene by Scorpio real time PCR. Assay Exon HM 3 was optimized for the analysis of a very short fragment within the GSTP1 gene with the use of Scorpion primers.

Methylation analysis of the GSTp1 gene by Scorpio real time PCR.

The scorpio real time PCR technology is used to investigate the methylation state of the the GSTp1 gene (see German patent application: 103 38 308.5; filing date: Aug. 15, 2003, applicant: Epigenomics AG). The following bisulfite treated fragment of the GSTp1 gene is amplified:

CGGGAttAtttTTATAAGGtTCGGAGGtCGCGAGGttTTCGtTGGAGTTTCGtCGtCGtAGT tTTCGttAttAG (nt 1845-nt 1924 in in Genbank Accession No. AY324387).

The PCR is conducted in a total volume of 20 μl. The reaction mix contains 10 μl of template DNA, 2 μl of FastStart LightCycler reaction mix for hybridization probes (Roche Diagnostics), 0.30 μM forward primer (GGGAttAtttTTATAAGGtT; SEQ ID NO:2), 0.10 μM reverse primer (TACTCACTaaTaaCKAAaACTaC; SEQ ID NO:38 ), 0.5 μM scorpion primer (FAM-ggcagccGtTGGAGtttCGtCGggctgcc-DDQ-HEG-TACTCACTAATAACKAAAACTAC; SEQ ID NO:XXX), 4 μM blocking probe (CTAATAACaAAAACTACaACaACaAAACTCCAAC-PHO; SEQ ID NO:48) and 3 mM MgCl₂. Instead of the above described standard Scorpio Primer, also duplex Scorpio primer can be used (FAM-GtTGGAGtttCGtCG-HEG-TACTCACTAATAACKAAAACTAC, Seq ID; CGaCGaaaCTCCAaC-DDQ; Seq ID NO:XXX; see German patent application: 103 38 308.5).

The reaction is performed using a Lightcyler device (Roche Diagnostics). The amplification is carried out under the following conditions: Thermocycling is beginning with a 95 degree C. incubation for 10 minutes followed by 55 cycles with following steps: 95 degrees C. for 10 seconds, 56 degrees C. for 30 seconds, and 72 degrees C. for 10 seconds. Fluorescence signals are detected prior to the annealing phase at 56 degrees C. in each cycle. The results show that the scorpio technology allows a specific detection of cytosine methylation within the GSTpi gene.

Due to the identical base pair behavior of uracil and thymine the positions corresponding to the converted (non methylated) cytosines were marked with a small “t” (resp. small “a” for the complementary strand). In contrast, “T” and “A” in capitals describe thymines already existing prior to the bisulfite treatment. The following abbreveations were used: FAM=fluoresceine label; DDQ: Deep Dark Quencher; DDQ: Deep Dark Quencher; HEG: hexaethylenglycol spacer; K: universal base dK

### DESCRIPTION OF THE FIGS. 1-4

FIG 1: Standard curve over 4 orders of magnitude.

The figure describes the standard curve of cycle threshold values that were determined over 4 orders of magnitude (62.5 pg to 100 ng). At the x-axis the logarithmic values of the amounts of template DNA are given. At the y-axis the mean out of four replicates of the threshold cycles number (Ct) as calculated by the LightCycler software is given. The threshold cycle number is a value that describes the number of PCR cycles that is necessary to give a sufficiently intense signal indicating the presence of the amplification product. It is the threshold cycle number that is used to calculate how much template DNA is detected in the tube. The formula calculated to describe said standard curve is: y=−3.3617x+34.081. The regression factor R² was calculated to be R²=0.9923.

FIG. 2: Real time quantitative HeavyMethyl assay on GSTP1 (exon 1).

The diagram in FIG. 2 shows the result of the quantitative HeavyMethyl assay (Exon HM 1) employing real time probes for analysis of the methylation pattern of the GSTP1 (exon 1) sequence as specified in the description. At the x-axis the number of PCR cycles is given. At the y-axis the levels of fluorescence (F2/F1) is indicated. The threshold cycle number (Ct) as calculated by the LightCycler software can be determined from such an output file. The different lines indicate the different experimental conditions with respect to the amount of background DNA. The GSTP1 (exon 1) specific HeavyMethyl assay was performed on 100 pg methylated bisulfite treated DNA (solid lines) and on 400 ng non-methylated bisulfite treated DNA (dotted lines; at the zero level). The 2 broken lines and the 2 lines labeled by circles show the performance of the assay at relative sensitivity values of 1:4000 and 1:8000, respectively.

FIG. 3: Real time quantitative HeavyMethyl assay (Exon HM 1) on GSTP1 (exon 1) in a duplex PCR approach, which simultaneously amplifies the GSTP1 (exon 1) fragment and a GSTP1 control fragment.

The diagram in FIG. 3 shows the result of a quantitative HeavyMethyl assay employing real time probes for detection of the methylated GSTP1 (exon 1) fragment and the GSTP1 control fragment as specified in the description. At the x-axis the number of PCR cycles is given. At the y-axis the levels of fluorescence (F2/F1) is indicated. The threshold cycle number (Ct) as calculated by the LightCycler software can be determined from such an output file. The selection of the fluorescence channel F2/F1 allows for the specific detection of the GSTP1 exon1 amplificate. The different lines indicate the different experimental conditions with respect to the amount of background DNA. The GSTP1 (exon 1) specific HeavyMethyl assay was performed on 1000 pg methylated bisulfite treated DNA (solid lines) and on 400 ng non-methylated bisulfite treated DNA (dotted lines; at the zero level). The lines labeled by circles show the performance of the assay using a mixture of 100 ng non-methylated and 500 pg methylated bisulfite treated DNA.

FIG. 4: Real time quantitative PCR assay (Exon HM 1) on GSTP1 control fragment in a duplex PCR approach, which simultaneously amplifies the GSTP1 (exon 1) fragment and a GSTP1 control fragment.

The diagram in FIG. 4 also shows the result of a quantitative HeavyMethyl assay employing real time probes for detection of the methylated GSTP1 (exon 1) fragment and the GSTP1 control fragment as specified in the description. At the x-axis the number of PCR cycles is given. At the y-axis the levels of fluorescence (F3/F2) is indicated. The threshold cycle number (Ct) as calculated by the LightCycler software can be determined from such an output file. The selection of the fluorescence channel F3/F2 allows for the specific detection of the GSTP1 control amplificate. The different lines indicate the different experimental conditions with respect to the amount of background DNA. The GSTP1 (exon 1) specific HeavyMethyl assay was performed on 1000 pg methylated bisulfite treated DNA (solid lines) and on 400 ng non-methylated bisulfite treated DNA (dotted lines). The lines labeled by circles show the performance of the assay using a mixture of 100 ng non-methylated and 500 pg methylated bisulfite treated DNA.

FIG. 5: Relative methylation values of prostate tumor tissue and BPH tissue in the GSTP1 exon 1 region analyzed by HM assay Exon HM 2.

A diagram is shown which presents at the x-axis the type of sample analysed (cancer or BPH, wherein BPH stands for benign prostate hyperplasia) and at the Y-axis the relative amount of methylation value in %.

1. A method for the detection of cytosine methylation in DNA samples, characterized in that the following steps are conducted:
a genomic DNA sample, which comprises the DNA to be investigated as well as background DNA is treated with bisulfite (═disulfite, hydrogen sulfite) in such a way that all of the unmethylated cytosine bases are converted to uracil, while the 5-methylcytosine bases remain unchanged;
the bisulfite treated DNA sample is amplified with the use of at least 2 primer oligonucleotides as well as a polymerase, wherein the DNA to be investigated is preferred over the background DNA as the template and
the amplification is conducted in the presence of at least one additional oligonucleotide or a PNA oligomer, which binds to a 5′-CG-3′ dinucleotide or a 5′-TG-3′ dinucleotide or a 5′-CA-3′-dinucleotide, whereby the other oligonucleotide or PNA oligomer preferably binds to the background DNA and adversely affects its amplification performed
and the methylation status in the DNA to be investigated is concluded from the presence and/or the amount of the amplified products and/or from the analysis of additional positions.

2. The method of claim 1 wherein a control fragment is amplified simultaneously to the amplification of the bisulfite treated DNA within the same reaction mixture.

3. The method of claim 1, characterized in that the DNA to be investigated comprises GSTP1 or its regulatory region.

4. The method according to claim 3 characterized by and employing thereby at least one primer out of the group consisting of SEQ ID NOs: 2, 42, 54, 58, 62 and 66 and one primer out of the group consisting of SEQ ID NOs: 3, 36, 38, 40, 43, 56, 60, 64 and 68 and at least one blocker out of the group of SEQ ID NO 4, 46, 48, 50, 52, 70, 72, 74, 76, 80, 81, 82, 83, 84, 85, 86, 87, 88, 89, 90, 91, 92, 93, 94, 95, 96, 97, 98, 99, 100, 102, 103 and SEQ ID NO: 104 is used.

5. The method according to claim 3, characterized in that at least one primer oligonucleotide from the group consisting of SEQ ID NOs: 2, 42, 54, 58, 62 and 66 and one primer oligonucleotide from the group SEQ ID NOs: 3, 36, 38, 40, 43, 56 and one additional oligonucleotide from the group consisting of SEQ ID NOs 4, 46, 48, 50, 52, 70, 72, 74 and 76 is used.

6. The method according to claim 1, further characterized in that the sample DNA is obtained from serum or urine or other bodily fluids of an individual.

7. The method according to claim 1, further characterized in that the DNA samples are obtained from cell lines, blood, sputum, stool, urine, serum, cerebro-spinal fluid, tissue embedded in paraffin, for example, tissue from eyes, intestine, kidneys, brain, heart, prostate, lungs, breast or liver, histological slides and all possible combinations thereof.

8. The method according to claim 1, further characterized in that said additional oligonucleotide is a ribonucleic acid oligonucleotide.

9. The method according to claim 1, further characterized in that the binding site of the additional oligonucleotide or PNA oligomer overlaps with the binding site of one of the primers on the background DNA and hereby hinders the binding of at least one primer oligonucleotide to the background DNA.

10. The method according to claim 1, further characterized in that the additional oligonucleotides and/or PNA oligomers are present in at least five times the concentration in comparison to the primer oligonucleotides.

11. The method according to claim 1, further characterized in that the additional oligonucleotides and/or PNA oligomers bind to the background DNA and thus hinder the complete elongation of primer oligonucleotides in the polymerase reaction.

12. The method according to claim 1, further characterized in that the chemically treated DNA sample is amplified in the second step with the use of at least 2 primer oligonucleotides and one additional oligonucleotide or PNA oligomer, which hybridizes to a 5′-CG-3′ dinucleotide or a 5′-TG-3′ dinucleotide or a 5′-CA-3′ dinucleotide, and at least one reporter oligonucleotide, which hybridizes to a 5′-CG-3′ dinucleotide or a 5′-TG-3′ dinucleotide or a 5′-CA-3′ dinucleotide, as well as a polymerase; whereby the additional oligonucleotide or PNA oligomer preferably binds to the background DNA and adversely affects its amplification, and whereby the reporter oligonucleotide preferably binds to the DNA to be investigated and indicates its amplification.

13. The method according to claim 1, further characterized in that different reporter oligonucleotides indicate the amplification of different products amplified simultaneously in one vessel.

14. The method according to claim 1 further characterized in that the additional reporter oligonucleotide indicates the presence of the control fragment.

15. The method according to claim 1, further characterized in that the reporter oligonucleotides are LightCycler probes, and a LightCycler assay is conducted.

16. The method according to claim 1, further characterized in that the reporter oligonucleotides by bearing at least one fluorescent label indicate the amplification either by an increase or a decrease in the fluorescence.

17. The method according to claim 1, further characterized in that the background DNA is present in 4000 times the concentration of the DNA to be investigated.

18. The method according to claim 1, further characterized in that the background DNA is present in 8000 times the concentration of the DNA to be investigated.

19. The method according to claim 1, characterized in that the control fragment is located within the same genomic region of the DNA to be investigated.

20. The method according to claim 1, further characterized in that the control fragment is located within a region of 2 kb upstream or 2 kb downstream of the CpG sites analysed.

21. The method according to claim 1, further characterized in that the control fragment is located within a region of maximum 1 kb upstream or maximum 1 kb downstream of the CpG sites analysed.

22. The method according to claim 1, characterized in that the genomic DNA to be investigated is the GSTP1 gene.

23. The method according to claim 1, characterized in that the genomic DNA to be investigated lies within bp 1183 and bp 1309 of the sequence defined by the Genbank Accession Number M24485.1.

24. The method according to claim 1, characterized in that the genomic DNA to be investigated lies within bp 1183 and bp 1309 of the sequence defined by the Genbank Accession Number M24485.1 and the control fragment lies within bp 2273 and bp 2303 of the sequence defined by the Genbank Accession Number M24485.1

25. A kit comprising at least one primer oligo nucleotide out of the group consisting of SEQ ID NOs: 2, 42, 54, 58, 62 and 66 and one primer (reverse) out of the group consisting of SEQ ID NOs: 3, 36, 38, 40, 43, 56, 60, 64 and 68 and at least one blocker out of the group of SEQ ID NO 4, 46, 48, 50, 52, 70, 72, 74, 76, 80, 81, 82, 83, 84, 85, 86, 87, 88, 89, 90, 91, 92, 93, 94, 95, 96, 97, 98, 99, 100, 102, 103 and SEQ ID NO:104.
